(19) United States
(12) Patent Application Publication
(10) Pub. No.: US 2024/0192116 A1
(43) Pub. Date: Jun. 13, 2024

(54) SYSTEMS AND METHODS FOR MICROSCOPIC OBJECT HANDLING

(71) Applicant: SCREENSYS GMBH, Freiburg i.Br. (DE)

(72) Inventors: Alexander GROSS, Ilmenau (DE); Jialan CAO, Ilmenau (DE); Johann Michael K¿HLER, Golmsdorf (DE); Steffen SCHNEIDER, Ilmenau (DE); Patrick SCHAUB, Freiburg i.Br. (DE); Klaus PALME, Freiburg i.Br. (DE)

(21) Appl. No.: 18/555,345

(22) PCT Filed: Apr. 14, 2022
(86) PCT No.: PCT/EP2022/060007

§ 371 Date: Oct. 13, 2023

(30) Foreign Application Priority Data
Apr. 15, 2021 (EP) 21168659.7
Mar. 7, 2022 (EP) 22160607.2

(51) Int. Cl. G01N 15/1409; B01L 3/00; G01N 35/08

(52) U.S. Cl. CPC G01N 15/1409; B01L 3/502761; G01N 35/08; B01L 2200/0647; B01L 2200/0673; B01L 2400/082

(57) ABSTRACT

System and method for handling dispersed microscopic objects contained in a sample fluid, the system comprising a first microfluidic device comprising a microchannel with an inlet, an outlet and an opening, the opening being located between the inlet and the outlet, a conveying device configured to pump a carrier fluid via the inlet into the microchannel with an input volumetric flow rate and to remove fluid from the microchannel via the outlet with an output volumetric flow rate, a first sensor unit for identifying positions of the dispersed objects in the sample fluid, and a positioning unit configured to position the opening at a target position proximate the position of a target object.

The present invention relates to systems and methods for handling microscopic objects, particularly the take-up and deposition of microscopic objects.

Repositioning microscopic objects, particularly microscopic objects in fluids, is a frequently occurring process for example in biological and biochemical processing, such as for repositioning biological and/or related objects, such as cells, embryos, bacteria, droplets, solid microparticles, gel microparticles, vesicles and/or parts of these.

Known techniques include two general strategies. The first strategy may be called “colony picking”, the second strategy may be called “single cell picking”.

Colony picking addresses and takes up cell aggregates or clusters. The addressed objects, namely the cell clusters/aggregates typically have dimensions in the order of approximately 0.1 mm to 1.0 mm. This technique allows for location-selective harvesting of portions of adhering cells, bacteria or the like and possibly for punching out pieces of tissue, e.g. for a biopsy.

Single cell picking addresses single cells having sizes in the order of approximately 1 μm to 100 μm by means of fine cannulas. Single cell picking systems typically take up single cells in cannulas or fix them to the tip of a cannula (e.g. patch-clamp technique), and reposition the single cells by (macroscopically) repositioning the cannulas for each cell or a limited number of cells, i.e. such a conventional system picks a cell with a cannula, repositions the cannula, deposits the cell at a target site and repeats these steps for all cells that are to be relocated. If necessary, the cannula is cleaned or exchanged between depositing one cell and taking up another cell. This is time and possibly material consuming.

The present invention is based on the concept of embedding microscopic objects in compartments or segments of a sample fluid, wherein the sample fluid compartments are embedded in another fluid called carrier fluid. This means that microscopic objects are embedded in segmented sample fluid flows.

Köhler et. al.: “Digital reaction technology by micro segmented flow-components, concepts and applications”, CHEMICAL ENGINEERING JOURNAL, (2004), vol. 101, pages 201-216, by co-inventors of the present invention, discloses segmented sample fluid flow, but does not address embedding further objects in the sample fluid segments.

Creating sample fluid compartments embedded in a carrier fluid is a frequently used technique in microfluidics. For example, WO 2010/142471 A1 and related EP 2 440 940 B1, by co-inventors of the present invention, disclose a device for generating and/or arranging sequences of a fluid sample in a carrier fluid. The device comprises a microchannel having an inlet, an outlet and a nozzle opening therebetween leading into the microchannel. These devices are used in a sample fluid containing three-dimensionally dispersed objects. The picking of individual and/or specific objects is generally not possible.

WO 2016/128544 A1 discloses a microfluidic probe head for providing a sequence of separate liquid volumes separated by spacers. In this probe head, liquid is ejected from a channel into an immersion liquid. It is believed that such arrangement may be disadvantageous, for example with regard to a possible wandering of target substances between different liquid volumes across the spacers.

Henkel et al.: “Chip modules for generation and manipulation of fluid segments for micro serial flow processes”, CHEMICAL ENGINEERING JOURNAL, (2004), vol. 101, pages 439-445, discloses sample generation by embedding sample liquid in a continuous stream of an immiscible carrier fluid.

Many other disclosures deal with the creation and/or manipulation of fluid segments, for example WO 2004/038363 A2; Ismagilov et al.: “Reactions and Droplets in Microfluidic Channels”, Angew. Chem. Int. Ed. 2006, 45, 7336-7356; Ismagilov et al.: “A Microfluidic Approach for Screening Submicroliter Volumes against Multiple Reagents by Using Preformed Arrays of Nanoliter Plugs in a Three-Phase Liquid/Liquid/Gas Flow”, Angew. Chem., 2005, 117, 2576-2579; and DE 103 22 893 A1, as summarized in WO 2010/142471 A1.

Further methods and devices are also known from, e.g., U.S. Pat. No. 5,080,866 A, JP 2008304270 A, EP 1 972 374 A2, and WO 2009/149257 A1 and PNAS 2008 105(44) 16843.

All above-mentioned disclosures are incorporated herein by reference in their entirety.

It is an object of the present invention to provide a system and method for selectively taking up one or more microscopic objects that are dispersed in a sample fluid. It is a further object of the present invention to provide a system and method for depositing one or more microscopic objects in corresponding sample fluid compartments, wherein the sample fluid compartments are embedded in a carrier fluid in a microchannel.

### SUMMARY

This object is achieved with the features of the independent claim(s). Dependent claims refer to preferred aspects of the invention.

According to a first aspect, the present invention relates to a system for handling dispersed microscopic objects contained in a sample fluid.

The objects may have a dimension or size of less than 1000 μm, preferably less than 500 μm, more preferably less than 100 μm, even more preferably less than 70 μm. The objects may have a size in the range of 10-1000 μm, preferably 10-100 μm, more preferably 30-70 μm. The term size may refer to a diameter, particularly a largest diameter or a mean diameter of an object. Alternatively, for example for strongly irregular shaped objects, the term size may refer in this context to the largest dimension of the object. Microscopic objects may include, for example, cells, particularly animal cells or insect cells and plant cells, bacteria, fungi, spores, plant seeds, plant pollen, spermatozoon, insect or animal eggs, droplets, vesicles, gel particles, and/or solid particles such as microbeads.

The system comprises a first microfluidic device and a conveying device. The first microfluidic device comprises a microchannel with an inlet, an outlet and an opening, the opening being located between the inlet and the outlet. The conveying device is configured to pump a carrier fluid via the inlet into the microchannel with an input volumetric flow rate Qᵢₙ and to remove fluid from the microchannel via the outlet with an output volumetric flow rate Qₒᵤₜ.

The cross section of the opening is selected such that, if the opening is in the sample fluid and the output volumetric flow rate Qₒᵤₜ is greater than the input volumetric flow rate Qᵢₙ, i.e. Qₒᵤₜ>Qᵢₙ, sample fluid enters the microchannel via the opening so that it is embedded as one or more compartments of sample fluid in the flow of the carrier fluid. Preferably, if the opening is in the sample fluid and the input volumetric flow rate Qᵢₙ is equal to the output volumetric flow rate Qₒᵤₜ, i.e. Qᵢₙ=Qₒᵤₜ, no carrier fluid emerges from the opening into the sample fluid and no sample fluid enters the microchannel.

The system is configured for a takeup mode in which, at least intermittently or continuously, the output volumetric flow rate Qₒᵤₜ is greater than the input volumetric flow rate Qᵢₙ, i.e. Qₒᵤₜ>Qᵢₙ, so that the system provides a flow of carrier fluid from the inlet through the microchannel and past the opening to the outlet and sequentially takes up one or more compartments of sample fluid into the flow of the carrier fluid with a takeup volumetric flow rate Qₜₐₖₑᵤₚ.

The system further comprises a first sensor unit for identifying positions of the dispersed objects in the sample fluid, including a position of a target object selected from the dispersed objects. The target object is an object that is to be taken up, in other words captured and/or embedded, in a takeup process.

The system further comprises a positioning unit configured to position the opening of the microfluidic device at a target position proximate to the position of the target object such that the target object is drawn into the microchannel together with a certain volume of sample fluid and thereby embedded with the sample fluid in the carrier fluid as a compartment. The positioning process may comprise a movement of the opening relative to a reference frame and/or of the target object relative to a reference frame.

In other words, when Qₒᵤₜ>Qᵢₙ, the difference in volumetric flow rates Qₒᵤₜ−Qᵢₙ results in a takeup volumetric flow rate Qₜₐₖₑᵤₚ of sample fluid through the opening into the microchannel. If the opening is positioned at the target position close to a target object, this results in the compartmentalization of the target particle in a compartment of sample fluid, the compartment of sample fluid being embedded in the flow of carrier fluid in the microchannel. The takeup causes a flow force that causes the carryover of the object into the compartment.

As surprisingly found by the inventors of the present invention, such arrangement allows the targeted reliable picking of determined microscopic objects despite their relatively small size (in comparison to the microfluidic device and its opening). By identifying the positions of target objects and repositioning the opening accordingly, the objects to be picked may be selected in a targeted manner and, surprisingly, without substantially disturbing their position.

The microchannel may have a cross section of at least 3000000 μm², at least 785000 μm², or at least 30000 μm². The microchannel may have a cross section of less than 3000000 μm², less than 785000 μm², or less than 30000 μm². The microchannel may have a radius of at least 1000 μm, at least 500 μm, or at least 100 μm. The microchannel may have a radius of less than 1000 μm, less than 500 μm, or less than 100 μm. For non-circular cross section shapes, the radii refer to the largest, the smallest or the mean radii. The microscopic objects may be cells, microorganisms, solid particles, gel particles, vesicles, droplets, embryos and/or parts thereof.

The opening (of the first and/or the second microfluidic device) may have a size of at least 785000 μm², at least 200000 μm², or at least 8000 μm². The opening may have a size of less than 800000 μm², less than 200000 μm², or less than 10000 μm². The opening may have a radius of at least 500 μm, at least 250 μm, or at least 50 μm. The opening may have a radius of less than 500 μm, less than 250 μm, or less than 60 μm. For non-circular cross section shapes, the radii refer to the largest, the smallest or the mean radii. The opening may have a cone-like or truncated cone-like shape, particularly the shape of a cone with an elliptical or circular base or the shape of a truncated cone with an elliptical or circular base. The opening may also have the shape of an elliptical or spherical triangle. The size of the opening may be defined as the cross section of the opening for fluid flow. The size of the opening may be adapted to the cross section of the microchannel. Preferably, the size of the opening is smaller or equal to the cross section of the microchannel in the region of the opening. A ratio of the size of the opening to the size of the cross section of the microchannel in the region of the opening may be 1.2 to 0.5, 1.1 to 0.7, or 1 to 0.9. The opening may be a nozzle opening.

The microchannel may have a cross sectional diameter of 500 μm and the opening may have a cross sectional diameter of 500 μm, which is suitable for takeup and deposition of objects with a size of 50 μm. This is particularly surprising because one would expect that the large (as compared to the object) opening and microfluidic devices would be too large handle and particularly to approach and take up such small objects that have a size of one or more orders of magnitude smaller than the channel.

In the region of the opening, for example in a region including the opening and 1 mm or 2 mm into each of the upstream and downstream directions, the direction of flow of the carrier fluid towards the opening and the direction of flow of the carrier fluid away from the opening may be parallel to each other (i.e. arranged at 180°), or they may be arranged at an angle of at least 80°, at least 90°, at least 110° or at least 130° to each other. This minimizes the risk of carrier fluid exiting the channel through the opening.

The system is preferably configured to take up several/a plurality of target objects in a corresponding number of sample fluid compartments. Preferably, the system is configured to take up the several target objects in immediately consecutive sample fluid compartments immediately consecutively embedded in the carrier fluid in the microchannel. In this manner, empty compartments may be avoided or at least their number may be minimized, which reduces subsequent post processing efforts such as sorting.

Preferably, the input volumetric flow rate Qᵢₙ of carrier fluid, the output volumetric flow rate Qₒᵤₜ of fluid and/or the takeup volumetric flow rate Qₜₐₖₑᵤₚ is/are continuous. Preferably, the settings and system properties are chosen such that the system is stable and/or the compartments are created at a frequency which remains constant over at least 10 seconds, at least 60 seconds, at least 10 minutes, or even longer.

Preferably, the geometries of the involved devices are chosen such that a perimeter of a sample fluid compartment at least fills the cross section of the microchannel. This contributes to a stable flow of sample fluid compartments in the carrier fluid in the microchannel without coalescence.

In the present invention, the carrier fluid is preferably a carrier liquid. The sample fluid is preferably a sample liquid. A support fluid (as further detailed below) may be a support liquid. The carrier liquid is preferably immiscible with the sample fluid/liquid. Immiscible means that two physical phases exist. The sample fluid and carrier fluid are immiscible at least in a way that minimum two liquid phases exist. Preferably, the sample liquid is as a hydrophilic liquid, preferable water or an aqueous solution. The sample liquid may be one or a combination of the following liquids: water, an aqueous solution such as pH-buffers, cell culture media or mixtures of water with polar organic solvents. Preferably, the carrier liquid is hydrophobic and immiscible with the sample liquid. The carrier liquid may be one or a combination of the following liquids: liquid alkanes such as tetradecane or mineral oils; aromatic solvents such as toluene; liquid oligo- or polysiloxanes; perfluorinated hydrocarbons such as perfluoromethyldecalin; aprotic organic solvents; non water miscible liquids which contain additives such as surfactants or modifiers adjusting the viscosity or surface tension; solutions of polymers or other substances, e.g. in one or a combination of the liquids mentioned herein. The carrier fluid may be a gas or may contain gas compartments in combination with other liquids, e.g. one of the liquids mentioned herein.

Preferably, the one or more target objects are trapped in the respective sample fluid compartments. This means that the system properties and settings are preferably chosen such that target object(s), once they have been taken up into the microchannel with a corresponding sample fluid compartment, do not exit their sample fluid compartment(s) to enter the carrier fluid and such that target object(s) do not switch between compartments. This means there is preferably essentially no object crosstalk among sample fluid compartments or between sample fluid compartments and carrier fluid. This may be the case even if the compartments flow upwards, i.e. against the force of gravity.

In order to achieve such conditions that essentially avoid object-crosstalk, one or more of the following conditions for the material defining the microchannel (also referred to as the “channel material” hereinafter), the carrier fluid and the sample fluid may be chosen:
The sample fluid wets the channel material less than the carrier fluid.
The sample fluid is an aqueous polar solvent, hydrophilic solvent, water, an aqueous solution and/or an aqueous mixture containing another solvent while the carrier fluid is non-miscible with the sample fluid, e.g. a liquid hydrocarbon or perfluoroinated hydrocarbonand the channel material, at least its inner surface, is hydrophobic and/or fluorophobic.
The carrier fluid is an aqueous polar solvent, hydrophilic solvent, water, an aqueous solution, and/or an aqueous mixture containing another solvent while the sample fluid is an organic nonpolar liquid, and the channel material or at least its inner surface is hydrophilic.

In order to adjust one or more properties of the involved materials/substances, one or more of the following options may be chosen:
The viscosities of the used fluids are adjusted by solution of one or more additives such as polymer(s) or inorganic compound(s).
The surface tension between the used fluids and/or the device surface is adjusted by addition of one or more surfactants such as sodiumdodecylsulfate or fluorosurfactant(s).
The surface wettability of the microfluidic device is adjusted by chemical modification of the surface using reactive compounds such as perfluorooctylsilan derivatives, polar modifying reagents and/or others.

The microscopic objects may be dispersed across a support surface. In other words, the system may be configured to take up microscopic objects dispersed across the support surface. Particularly, the shape of the first microfluidic device may be configured for the take up of microscopic objects dispersed across the support surface. Preferably, the support surface is the bottom surface of a container in which the sample fluid is contained, for example a Petri dish, a well of a well plate, a cuvette, an object slide, a cover slip, or the like. Preferably, the microscopic objects are sedimented to the support surface. Preferably, sedimentation of the objects occurs under gravity of planet Earth. Dispersing the microscopic objects across a support surfaces simplifies identification of their positions and stabilizes said positions. Moreover, movement of the objects when moving the microfluidic device through the sample fluid is reduced at least to some extent. The system may comprise the support surface and/or container.

The first sensor unit may be configured for generating corresponding sensor data.

The first sensor unit may comprise a first imaging unit configured for imaging the dispersed objects and preferably for creating corresponding imaging data. For example, the first imaging unit may be configured for light microscopy, particularly bright field microscopy, dark field microscopy, phase contrast microscopy, confocal microscopy, fluorescence microscopy and/or differential interference contrast microscopy. For example, the first imaging unit may comprise a lamp and an objective for collecting light from the illuminated microscopic objects. The lamp may illuminate the objects from a lateral direction i.e. obliquely or perpendicular to an imaging axis (e.g. the optical axis of a light collecting means) of the imaging unit. This may be advantageous when a scattered light signal is relied upon, e.g. with fluorescent objects. Preferably a scatter lighting arrangement is used to detect scattered light from the target object at reduced background.

The system may comprise a second sensor unit configured for analyzing the target object while embedded in the carrier fluid in the microchannel, i.e. while embedded in a compartment of sample liquid embedded in the carrier fluid in the microchannel. In general, the wording “target particle embedded in the sample fluid” includes all possible locations of the target particle as long as the target particle is in contact with the sample fluid. For example, the target being located at the very edge of the sample fluid compartment would still be called “embedded”. Preferably the second sensor unit is configured for generating corresponding sensor data.

The second sensor unit may be used to determine additional spectral properties of the compartments of sample fluid and/or the target object itself. For example, the second sensor unit may be configured to determine color, optical density, spectral absorption, transparency, fluorescence, phosphorescence, Raman resonances. The second sensor unit preferably comprises a second imaging unit configured for imaging the target object while embedded in the carrier fluid in the microchannel, i.e. while the microscopic object is embedded in a compartment of sample liquid embedded in the carrier fluid in the microchannel. Preferably, the second imaging unit is configured for creating corresponding imaging data. The second imaging unit may be configured for light microscopy, particularly bright field microscopy, dark field microscopy, phase contrast microscopy, confocal microscopy, fluorescence microscopy and/or differential interference contrast microscopy. Preferably fluorescence microscopy is used to quantify fluorescence intensity of the target object. Alternatively, or additionally, the second sensor unit may comprise a light emitting diode and a light detector configured for detecting the light emitted from the diode that passed through the microchannel in a time resolved manner. This enables analysis of the size of the compartments of sample fluid embedded in the carrier fluid and enables process control. The second unit may be configured as a spectral analyzing unit (i.e. spectroscopic unit) generating spectroscopic data. Spectroscopy may comprise UV-Vis spectroscopy, fluorescence spectroscopy, phosphorescence spectroscopy, Infrared and/or Raman-Spectroscopy, and respective spectroscopic data may be created, stored and/or further processed.

The first microfluidic device may comprise at least one optical element, preferably for the second sensor unit. The at least one optical element is preferably a lens, a glass window, an aperture, an optical lattice and/or a wave guide. The at least one optical element may additionally or alternatively be configured to couple light in and out for different purposes. For example, a laser may be coupled in, wherein the laser is configured for manipulating at least some of the microscopic objects prior to, during and/or after takeup into the microchannel. The laser may be configured to be focused to the support surface and to separate clustered microscopic objects into dispersed microscopic objects. Alternatively or additionally, the laser may be used for optically activating photoactivatable substances in the microchannel, i.e. in the sample fluid, carrier fluid, the target object and/or other fluids and/or material added to the microchannel as described elsewhere herein. The optical element(s) may be arranged for locally focused fluorescence spectroscopy wherein the target is excited with focused light and fluorescence light is collected.

Optionally, the second imaging unit is provided proximate the opening.

Alternatively or additionally, the system may comprise one or more optical elements, for example one or more lasers, configured for manipulating at least some of the microscopic objects prior to takeup into the microchannel, wherein the light is not coupled into and/or does not run through the microchannel.

The microchannel may comprise an additional inlet, wherein the system is configured to deliver a fluid, preferably carrier fluid, into the microchannel via the additional inlet at an additional volumetric flow rate Q_{add}.

The system may be configured to deliver the fluid via the additional inlet intermittently into the microchannel at determined time points. This may serve different purposes. For instance, if additional carrier fluid is added to the microchannel via the additional inlet, this reduces the takeup volumetric flow rate Qₜₐₖₑᵤₚ accordingly and may consequently inhibit takeup of sample fluid. With set values of Qₒᵤₜ and Qᵢₙ, the sample liquid compartments are embedded in the carrier liquid with regular spacing. In case the system is to takeup two target particles in two immediately consecutive compartments of sample fluid, the time necessary for moving the opening from the first target particle to the second target particle may be too long to takeup the two target particles in two immediately consecutive sample liquid compartments. In a system without the additional inlet, this could result in a first compartment comprising the first target particle, followed by a compartment of sample liquid without any target particle, followed by a compartment of sample liquid comprising the second target particle. With the additional inlet, the system may be configured to avoid such sample liquid compartments without any target particle by adding additional fluid to the microchannel to delay the formation of a sample fluid compartment until the opening has reached a target position.

Particularly, the microchannel of the first microfluidic device may comprise the additional inlet for additionally providing a fluid, preferably a liquid and more preferably the carrier fluid or a another carrier fluid that is immiscible with the sample fluid, via the additional inlet to the microchannel at an additional volumetric flow rate Q_{add}. The takeup mode may comprise, at least intermittently:
the input volumetric flow rate Qᵢₙ and the output volumetric flow rate Qₒᵤₜ are kept constant;
the additional volumetric flow rate Q_{add} is kept smaller than the absolute value of the difference |Qᵢₙ−Qₒᵤₜ| of the input volumetric flow rate Qᵢₙ and the output volumetric flow rate Qₒᵤₜ, preferably zero, for the takeup of target objects, and preferably also when approaching the opening to the target objects, to allow for takeup of sample fluid through the opening of the first microfluidic device; and
wherein otherwise the additional volumetric flow rate Q_{add} is set to be essentially equal to the absolute value of the difference |Qᵢₙ−Qₒᵤₜ| of the input volumetric flow rate Qᵢₙ and the output volumetric flow rate Qₒᵤₜ to prevent takeup of sample fluid through the opening.

Alternatively or additionally, the takeup mode may comprise: keeping the output volumetric flow rate Qₒᵤₜ constant and intermittently varying the input volumetric flow rate Qᵢₙ in order to prevent the formation of one or more compartments of sample fluid when the opening is not proximate to a particle or one or more particles proximate to the opening are not to be drawn into the microchannel. The system may be configured to vary the input volumetric flow rate Qᵢₙ by a rapid action valve.

The microchannel may comprise a manipulation inlet arranged at the opening or between the opening and the outlet. The system may be configured to deliver a fluid (also referred to as a “manipulation fluid” herein) via the manipulation inlet into a respective compartment in the microchannel at a manipulation volumetric flow rate Qₘₐₙᵢₚ. The manipulation fluid may be miscible with the sample fluid. Miscible in this context means the fluid can be mixed homogenous with the sample fluid. The manipulation fluid preferably is immiscible with the carrier fluid. The manipulation fluid may be a hydrophilic liquid, preferable water, an aqueous solution and/or aqueous solvent mixture.

The manipulation fluid may be selected from: a gas to be metabolized by the objects, a liquid containing a growth stimulator for the objects, a liquid containing one or more type of bacteria, a liquid containing culture media composed of nutrition and/or pH-Buffer components, a liquid containing an enzyme, a liquid containing ingredients for digestion of the object, parts of the object or liberation of surface bound molecules, a liquid containing compounds for analytical assay purposes, a liquid containing a PCR mix, a liquid containing antibodies, a liquid containing nanomaterial(s) preferably for surface-enhanced Raman spectroscopy, a liquid containing particles, and/or a liquid containing marker substances, preferably dyes.

The first microfluidic device may comprise a tube, the tube defining the microchannel and the opening, such as a flexible tube made from a polymeric material. The tube may be made, for example, from polyamide, polypropylene, polyethylene, polyvinylchloride, polysiloxanes and/or fluorinated polymers such as polytetrafluorethylene (PTFE), perfluorethylenpropylen (FEP), Perfluoroalkoxyalkane (PFA) or other of that kind.

The tube preferably comprises a loop section and the opening is preferably located at an outer circumference of the loop section. The position of the opening in the tube is preferably oriented towards a target object, when the loop/opening is moved towards the target object. The loop section may have a radius of less than 5 mm, preferably less than 1 mm.

The first microfluidic device may alternatively comprise a microfluidic chip, preferably a microfluidic glass or polymer chip, the microfluidic chip defining the microchannel and the opening.

The system may comprise a container in which the sample fluid is contained. Preferably, the container comprises the support surface.

The container and/or the support surface may comprise a structure for prearranging the microscopic objects, preferably a depression or a pattern of depressions, for example one or more elongated grooves and/or one or more wells. The one or more elongated grooves may have a width of less than 200 μm, preferably less than 100 μm. The one or more wells may have a maximum width or a diameter of less than 200 μm, preferably less than 100 μm.

Such a prearranging of the microscopic objects helps in optimizing the takeup process. For example, the microscopic objects are easier to find and detect by the first detection unit due to the microscopic objects being confined according to the structure. Moreover, the area covered by and the distances between microscopic objects, and thus the potential target objects, are predetermined, such that the total time needed for analyzing the microscopic objects and takeup of one or more target objects can be reduced and/or the travel path of the opening can be optimized.

The system may further comprise a tip and/or protrusion that is positioned proximate to and/or at least partially around the opening. The tip may be configured for contacting a support surface on which the objects are dispersed in order to separate the target object from other dispersed objects and/or to avoid other dispersed objects than the target object from being drawn into the microchannel together with the respective compartment of sample fluid. Preferably, the tip is tapered and/or terminates at an edge.

The system may be configured for storage, incubation, cultivation, transport and/or preparation of the target objects that have been taken up.

The system may comprise a storage configured to accommodate the target object(s) while embedded with the sample fluid in the carrier fluid for storage, cultivation and/or further treatment of the target object(s).

Preferably, the first microfluidic device is microfluidically connected to the storage and or comprises the storage. With such system, a very large number, for example at least 100, at least 500, or at least 1000, or several thousand sample fluid compartments embedded in carrier liquid, and thus target objects embedded in the sample fluid compartments, may be taken up. Moreover, the target objects may be taken up from a three dimensional, two dimensional or one dimensional sample and be arranged at defined, one dimensional positions, i.e. in a row of sample liquid compartments.

The storage may comprise, preferably be, a microchannel, such as a tube (e.g., a fluoropolymer tube) or chip device with e.g. meandered microchannel. The microchannel or tube may be at least 5 cm, preferably at least 50 cm or at least 5 m long or may have any suitable length.

The system may comprise a microfluidic treatment unit. Such a microfluidic treatment unit may comprise any microfluidic structure for manipulating the fluids and compartments as received from the microchannel as known in the art. Compartment manipulating operations may comprise the addition of fluids or aliquotation of the compartment content, splitting or merging of compartments, fusion with other compartment series containing reagents, stimulating chemicals, tissue fragments, sphaeroides, microorganisms, viruses, cells or other biological microobjects.

According to a further aspect, which may be provided in conjunction with the first aspect described above but also employed separately therefrom, the present invention relates to a system for deposition of microscopic objects. In particular, the system may be configured to deposit one or more microscopic objects embedded with a sample fluid in a carrier fluid at one or more corresponding target sites.

The system may comprise a microfluidic deposition device for depositing the one or more microscopic objects at the target sites, wherein the deposition device comprises a microchannel with an opening and at least one inlet, wherein a plurality of compartments of sample fluid embedded in a carrier fluid are flown via the inlet to the opening, wherein at least some of the compartments of sample fluid are dispensed through the opening and deposited on a target surface.

The microchannel of the deposition device may be further provided with an outlet, wherein a volumetric flow rate through the inlet of the deposition device when dispensing a compartment of sample fluid Qᵢₙ is greater than a volumetric flow rate through the outlet of the deposition device Qₒᵤₜ when dispensing the compartment of sample fluid. Optionally, flow through the outlet of the deposition device is blocked or reduced for dispensing the compartment of sample fluid.

The microchannel of the deposition device may be further provided with a manipulation inlet, wherein the system is configured to deliver a support fluid via the manipulation inlet of the deposition device into the microchannel of the deposition device, preferably at determined time points, to squeeze a compartment of sample fluid located proximate to the opening of the deposition device out of the microchannel of the deposition device through the opening of the deposition device.

The manipulation inlet of the deposition device may be arranged at the opening of the deposition device, between the inlet and the opening of the deposition device, or between the opening and an outlet of the microchannel of the deposition device.

The deposition device may be provided by a second microfluidic device, preferably a second microfluidic device that is microfluidically connected to the first microfluidic device.

Alternatively, the deposition device may be provided by and preferably be the first microfluidic device, i.e. such that the microchannel of the deposition device is the microchannel of the first microfluidic device, and the opening of the deposition device is the opening of the first microfluidic device.

Several configurations are contemplated in this respect. In a first configuration, the inlet and the outlet of the microchannel of the first microfluidic device in the takeup mode are also the inlet and the outlet of the microchannel in the deposition mode. In this case, the system is configured to guide or convey the sample fluid compartments from the outlet of the microchannel to the inlet of the microchannel. For example, the deposition may occur in the order of takeup. Of course, e.g. by means of a device for microfluidic rearrangement, the deposition of sample fluid compartments may also occur in the reversed order of takeup.

In a second configuration, the system is configured to operate at a reversed flow direction of carrier fluid in the deposition mode as compared to the takeup mode. In the second configuration, the system is configured to take up several sample fluid compartments, to reverse the flow direction of carrier fluid and thereby switch inlet and outlet of the microchannel upon switching from takeup mode to deposition mode, and to deposit the sample fluid compartments. For example, the deposition may occur in reversed order of takeup. Of course, e.g. by means of a device for microfluidic rearrangement, the deposition of sample fluid compartments may also occur in the order of takeup.

In a further configuration, in addition to or alternatively to guiding/conveying the sample fluid compartments back to the inlet of the takeup mode, any other inlet of the microchannel, such as the ones described above, may be used for providing the sample fluid compartments to the opening in the microchannel. In other words, the additional inlet or the manipulation inlet of the takeup mode could equally form the inlet of the device during deposition.

The system preferably comprises a target surface for depositing the object(s). The target surface preferably comprises areas that are more wettable by the sample fluid than by the carrier fluid. In this context, the term “not wettable” means that the respective liquid forms a contact angle θ of at least 90°, at least 100°, or at least 120° on the respective surface and “wettable” means that the respective liquid forms a contact angle θ of less than 90°, preferably less than 60°.

The target surface may comprise a pattern of areas wettable by the sample fluid and one or more areas non-wettable by the sample fluid. The target sites may correspond to the wettable areas.

Particularly, the wettable areas may be wettable by the sample fluid and the non-wettable areas may be less wettable by the sample fluid. Preferably the wettable areas are less wettable by the carrier fluid and/or a support fluid than by the sample fluid. More preferably, the non-wettable areas are not wettable by the sample fluid and/or the support fluid.

The wettable areas may be patches that are, preferably regularly, arranged on the otherwise non-wettable target surface (e.g., neither wettable by the carrier fluid nor by the sample fluid). In other words, the otherwise non-wettable target surface may comprise one or more, preferably regular, arrays of wettable areas.

The wettable areas may comprise glass or activated silicon surfaces, hydrophilic polymers, hydrophilized surfaces by oxygen plasma or surface wet etching or laser ablation, functionalized metal or ceramics surfaces. The non-wettable areas may comprise hydrophobic polymer surfaces, hydrophobized silicon, glass, ceramics or metal surfaces and/or not be hydrophilized.

The system may be configured to change a relative position of the opening of the deposition device to the target surface, preferably via the positioning unit.

The system may be configured to change the relative position of the opening of the deposition device to the target surface continuously, preferably at a constant velocity, and more preferably at a velocity adapted to a pattern of areas wettable by the sample fluid. Even more preferably, the volumetric flow rate through the inlet when dispensing a compartment Qᵢₙ is constant.

The target surface may comprise areas, preferably target sites, that are more absorbent for the sample fluid than for the carrier fluid or vice versa. The target surface may comprise areas, preferably target sites, that are absorbent for both the sample fluid and the carrier fluid. Optionally, the absorbent/more absorbent areas correspond to the wettable areas. Absorbent in this context means the fluids are moved into the material by a capillary wicking effect and dispersed objects remain on the surface. The absorptive material may comprise paper, cotton, textile mesh, dry gel-materials, inorganic materials and/or microstructure materials wich possess capillary wicking effect for one or more of the used fluids. These areas may be arranged in a, preferably regular, pattern. The target surface may comprise a pattern of areas absorbing sample fluid and one or more areas not absorbing sample fluid. The target sites may correspond to areas absorbing sample fluid.

The system according to any of the above-mentioned aspects may comprise a control unit configured to control the conveying device, preferably in order to vary the ratio between the input volumetric flow rate Qᵢₙ and the output volumetric flow rate Qₒᵤₜ. The control unit may be and/or comprise any suitable device or combination of devices, such as a microcontroller, a PC, etc.

The control unit may be configured to, preferably automatically, control movement of the first and/or second microfluidic devices, and/or takeup, storage, culturing, treatment and/or deposition of the one or more objects.

The control unit is preferably configured to identify and/or appraise one or more objects, preferably one or more target objects, among the dispersed objects from the first sensor data created by the first sensor unit, preferably from the first imaging data created by the first imaging unit. For this and other purposes, the control unit may be configured to employ an object recognition algorithm, for example an artificial intelligence object recognition algorithm. Additionally or alternatively, a user may provide criteria that are used by the system to identify the one or more target objects. Such provision of selection criteria by a user may be optional in the system, i.e. if a user provides selection criteria, the system uses these criteria. If a user does not provide selection criteria, the system may perform a default procedure. Additionally or alternatively, a user may have the option to manually select one or more target objects among the dispersed objects.

The control unit is preferably configured to determine and/or execute a takeup sequence automatically. A takeup sequence is a sequence of steps performed by the system in order to takeup target particles. The takeup sequence may comprise a sequence of steps with one or more steps selected from: creation of sensor data by the first sensor unit, preferably imaging data from the first imaging unit; identification of one or more target objects among the dispersed objects from the first sensor data created by the first sensor unit; calculation of target positions corresponding to the target objects; calculation of a, preferably optimized, path of the opening of the first microfluidic device to reach all target objects, i.e. to reach all target positions; calculating, preferably optimized, settings for all required volumetric flow rates; setting the system to the takeup mode; applying the respective settings to the conveying device; moving the opening of the first microfluidic device along the calculated path thereby taking up the target particles in corresponding sample fluid compartments embedded in the carrier fluid; surveying the uptaken sample fluid compartments with the second sensor unit, preferably the second imaging unit, thereby creating second sensor data, preferably second imaging data.

The system may be configured to move the opening at a velocity v_{opening} towards the target object, wherein the velocity v_{opening} is selected such that the takeup volumetric flow rate Qₜₐₖₑᵤₚ compensates, at least partially compensates, or substantially compensates for a displacement of sample fluid towards the target object caused by moving the first microfluidic device through the sample fluid.

The system's takeup mode may, at least intermittently, comprise: the control unit is configured to keep the takeup volumetric flow rate Qₜₐₖₑᵤₚ constant and the velocity at which the opening is moved v_{opening} between a plurality of consecutive target objects is adjusted according to a distance between each pair of consecutive target objects. Keeping the takeup volumetric flow rate constant while adjusting the velocity may be favorable since an adjustment of the input and/or output volumetric flow rate may affect the takeup volumetric flow rate only with substantial delay. Preferably, the plurality of consecutive target objects are sequentially drawn into the microchannel with respective, equally spaced compartments of sample fluid. Preferably, there are no sample fluid compartments without a target particle between sample fluid compartments with a target particle, i.e. the system is configured to create a sequence of sample fluid compartments, wherein every sample fluid compartment comprises a target particle.

At least intermittently, the takeup mode may comprise: the takeup volumetric flow rate Qₜₐₖₑᵤₚ is kept constant, the velocity of the opening v_{opening} is kept constant, and a travel path of the opening between a pair of consecutive target objects is adapted accordingly to a distance between the pair of target objects. Preferably, the pair of target objects are sequentially drawn into the microchannel with respective, consecutive, equally spaced compartments of sample fluid. In this case, in which the takeup volumetric flow rate Qₜₐₖₑᵤₚ is kept constant, the velocity of the opening v_{opening} is kept constant and the path of the opening is adapted to the velocity of the opening v_{opening} and the distances between the target particles such that the arrival of the opening at the target sites is just in time for takeup of the target particles in regularly spaced sample fluid compartments that preferably all comprise a target particle.

The system may comprise an empty compartment identification means for automatically identifying compartments of sample fluid in which no object is contained. The empty compartment identification means may comprise the second sensor unit, preferably the second imaging unit, or vice versa. The control unit may be configured to control the empty compartment identification means and/or to process data created by the empty compartment identification means.

According to further aspects, the present invention also relates to methods for handling microscopic objects. The methods may be implemented by using the systems described above. The features of the systems may translate to features of the methods and vice versa. In general, all method steps may be at least partially performed manually by a user and all method steps may be at least partially performed automatically by the systems as described above. It is contemplated that some of the steps are performed at least partially manually while some of the steps are performed automatically.

According to one such method aspect, the present invention relates to a method for picking microscopic objects by suction. The method may comprise the steps:
a) providing a sample of dispersed microscopic objects contained in a sample fluid;
b) providing a first microfluidic device comprising a microchannel with an inlet, an outlet and an opening located between the inlet and the outlet;
c) providing a carrier fluid that is immiscible with the sample fluid via the inlet to the microchannel at an input volumetric flow rate Qᵢₙ and removing fluid from the microchannel via the outlet at an output volumetric flow rate Qₒᵤₜ;
d) positioning the opening in the sample fluid;
e) setting the input volumetric flow rate Qᵢₙ to be smaller than the output volumetric flow rate Qₒᵤₜ, the difference resulting in a takeup volumetric flow rate Qₜₐₖₑᵤₚ of sample fluid through the opening that results in compartments of sample fluid embedded in the carrier fluid;
f) changing a position of the opening relative to a target object from a starting relative position to a target position to bring the opening proximate to the target object;
g) drawing the target object into the microchannel together with a respective compartment of sample fluid when embedding the compartment of sample fluid in the carrier fluid according to step e).

A velocity v_{opening} at which the position of the opening relative to a target object is changed in step f) may be selected such that the takeup volumetric flow rate Qₜₐₖₑᵤₚ compensates or substantially compensates for a displacement of sample fluid towards the target object caused by moving the first microfluidic device through the sample fluid.

Steps f) and g) may be performed for a first target object and repeated for a second target object while keeping the takeup volumetric flow rate Qₜₐₖₑᵤₚ constant, wherein the velocity of the opening v_{opening} is adjusted according to the distance between the first and second target objects to embed the first and second target objects with consecutive compartments of sample fluid, preferably with immediately consecutive and equally spaced first and second compartments of sample fluid, respectively.

The steps f) and g) may be repeated while keeping the takeup volumetric flow rate Qₜₐₖₑᵤₚ constant, wherein the velocity of the opening v_{opening} is adjusted accordingly for each pair of immediately consecutive target objects to sequentially embed several target objects with a corresponding number of compartments of sample fluid, preferably with a corresponding number of consecutive, equally spaced compartments of sample fluid, more preferably with a corresponding number of immediately consecutive, equally spaced compartments of sample fluid.

The steps f) and g) may be performed for a first target object and repeated for a second target object while keeping the takeup volumetric flow rate Qₜₐₖₑᵤₚ constant, wherein the travel path of the opening between the first and second target objects is adapted to embed the first and second target objects with consecutive compartments of sample fluid, preferably with immediately consecutive first and second compartments, respectively. The velocity of the opening v_{opening} may be kept constant.

For example, the travel path between a pair of immediately consecutive target objects may differ from a straight line (e.g., it may be angled or curved). The travel path may deviate from a straight line parallel to a support surface in lateral directions parallel to the support surface and/or upwards and downwards. In this context, upwards and downwards is to be understood as away from the support surface and towards the support surface.

The method steps f) and g) may be repeated for several target objects while keeping the takeup volumetric flow rate Qₜₐₖₑᵤₚ constant, wherein the velocity of the opening v_{opening} is kept constant and wherein the travel path of the opening between the first and second target objects of each pair of consecutive target objects is adapted to sequentially embed several target objects with a corresponding number of compartments of sample fluid, preferably with a corresponding number of consecutive, equally spaced compartments of sample fluid.

Alternatively or additionally the method may include adjusting the input and/or the output volumetric flow rate, and/or delivering additional fluid into the channel. For example, the microchannel may comprise an additional inlet for additionally providing a fluid that is immiscible with the sample fluid via the additional inlet to the microchannel at an additional volumetric flow rate Q_{add}, preferably carrier fluid. Preferably, the equation Qₒᵤₜ=Qᵢₙ+Qₜₐₖₑᵤₚ+Q_{add} applies. The additional volumetric flow rate Q_{add} may be set to be smaller than the absolute value of the difference |Qᵢₙ−Qₒᵤₜ| of the input volumetric flow rate Qᵢₙ and the output volumetric flow rate Qₒᵤₜ, preferably zero, for step g), and preferably for arrival of the opening at the target position in step f), to allow for takeup of sample fluid through the opening, whereas otherwise the additional volumetric flow rate Q_{add} may be set to be essentially equal to the absolute value of the difference |Qᵢₙ−Qₒᵤₜ| of the input volumetric flow rate Qᵢₙ and the output volumetric flow rate Qₒᵤₜ to prevent takeup of sample fluid through the opening. The input volumetric flow rate Qᵢₙ and the output volumetric flow rate Qₒᵤₜ could be kept constant.

The method may further comprise the steps of analyzing at least some or each compartment of sample fluid, preferably optically, and comparing the, preferably optical, analysis result with a predefined standard, thus creating a labeling result for each compartment, and labeling each compartment according to the labeling result.

The method may also include providing the sample in a container, wherein the objects are located on a surface of the container, preferably on a bottom surface of the container.

As described for the first microfluidic device above, the microchannel may comprise a manipulation inlet arranged at the opening or between the opening and the outlet. The method may further comprise in this case delivering a manipulation fluid via the manipulation inlet into a compartment in the microchannel at a manipulation volumetric flow rate Qₘₐₙᵢₚ. Preferably, the equation Qₒᵤₜ=Qᵢₙ+Qₜₐₖₑᵤₚ+Qₘₐₙᵢₚ+Q_{add} is fulfilled. If Q_{add}=0, for example due to respective regulation or due to an additional inlet not being present, the equation may be simplified to read Qₒᵤₜ=Qᵢₙ+Qₜₐₖₑᵤₚ+Qₘₐₙᵢₚ. In general, the balance of entering and leaving volumes must be correct. Qᵢₙ, Qₒᵤₜ, Qₘₐₙᵢₚ and Q_{add} may be actively controlled, Qₜₐₖₑᵤₚ may automatically adjust accordingly.

Additionally or alternatively, the sample fluid may be a liquid and the manipulation fluid may be selected from at least one of: a liquid miscible with the sample liquid (e.g., a liquid containing a growth stimulator for the objects, or a liquid containing one or more type of bacteria) and a gas to be metabolized by the objects.

The method may comprise the step: prearranging the objects in a specific pattern for takeup, e.g. in one or more lines.

The method may comprise the steps: detecting positions of at least some of the objects in the sample, preferably optically; and selecting the target objects(s) among the objects for which the position was detected. The step of selecting the target object(s) may comprise the step of identifying and excluding objects that are unsuitable. The step of selecting the target object(s) may comprise the step of identifying and excluding groups of objects in which the objects are determined to be too close to each other, such as clusters or aggregates of objects. Preferably, groups of objects in which the objects are determined to be too close to each other for embedding them individually into respective compartments of sample fluid are excluded. Identifying objects, excluding objects and/or selecting the target objects(s) optionally comprises using artificial intelligence.

According to a further method aspect, the present invention relates to a method for depositing microscopic objects that are embedded with a sequence of compartments of a sample fluid in a carrier fluid. The method for depositing may be used for deposition the objects picked by suction with the method as described above. The skilled person will thus appreciate that both methods may be provided independently but could also form part of a single method for picking and depositing the objects.

Particularly, the present invention relates to a method for depositing a sequence of microscopic objects that are embedded with a sequence of compartments of a sample fluid in a carrier fluid, the carrier fluid being immiscible with the sample fluid, the method comprising the steps:
i) providing
   a target surface that is wettable with the sample fluid and/or absorbent for the sample fluid, preferably wherein the surface is absorbent for the carrier fluid, or preferably wherein the surface is poorly wettable with and/or poorly absorbent for the carrier fluid; or
   a target surface that is absorbent for the carrier fluid but poorly absorbent for the sample fluid; or
   a target surface that is less absorbent for the sample fluid than for the carrier fluid; or
   a target surface that is wettable with the sample fluid and/or absorbent for the sample fluid and wherein the surface is less wettable with and/or less absorbent for the carrier fluid;
j) providing a microchannel having an inlet and an opening through which the objects are dispensed;
k) providing the sequence of microscopic objects embedded with the sequence of compartments of the sample fluid in the carrier fluid in the microchannel;
l) moving the opening relative to the target surface, thereby positioning the opening at a target position relative to a target site on the target surface, the target position allowing for drops emerging from the opening to contact the target surface when still in contact with a wall of the microchannel and/or the fluid in the microchannel;
m) flowing the sequence of compartments through the microchannel towards the opening and dispensing the compartment closest to the opening in the direction of flow through the opening at the target position;
n) repeating steps l) and m) in order to deposit microscopic objects embedded with different compartments of sample fluid at different target sites.

The opening may be moved during step m) in order to help separation of one compartment and/or its surrounding carrier fluid.

In the method of the invention, the microchannel further comprises an outlet and the opening is located between the inlet and said outlet, the method further comprising the following steps:
providing fluid via the inlet to the microchannel at an input volumetric flow rate Qᵢₙ and simultaneously removing fluid from the microchannel via the outlet at an output volumetric flow rate Qₒᵤₜ;
wherein the input volumetric flow rate Qᵢₙ is larger than the output volumetric flow rate Qₒᵤₜ, the difference resulting in a discharge volumetric flow rate Q_{deposit} of sample fluid through the opening that results in deposition of the compartments of sample fluid embedded in the carrier fluid at the target site.

The repeated positioning of the opening may be performed by a continuous movement of the opening, preferably at a constant velocity.

The target surface may be any of the target surfaces mentioned herein. Particularly, the target surface may comprise a pattern of areas wettable with and/or absorbent for the sample fluid and areas non-wettable with and/or non-absorbent for the sample fluid and the target sites may correspond to the areas wettable with/absorbent for the sample fluid. The target surface and particularly said areas that are wettable with/absorbent for the sample fluid may be absorbent for the carrier fluid. The target surface may comprise a pattern of areas absorbent for the carrier fluid but poorly/less absorbent for the sample fluid and the target sites may correspond to these areas.

The repeated positioning of the opening may be a continuous movement of the opening, preferably at a constant velocity, and preferably adapted to a pattern of wettable and non-wettable areas of the target surface.

The wettable areas may be patches that are, preferably regularly, arranged on the otherwise non-wettable target surface.

If the microchannel further comprises a manipulation inlet, the method may comprise the step of delivering a supporting fluid via the manipulation inlet into the microchannel at or proximate to the compartment closest to the opening in the direction of flow at determined time points to dispense the compartment of sample fluid closest to the opening in the direction of flow through the opening.

The method may include delivering the fluid through the manipulation inlet at a manipulation volumetric flow rate Qₘₐₙᵢₚ, wherein Qₒᵤₜ=Qᵢₙ+Qₘₐₙᵢₚ−Q_{deposit}.

Information on sample fluid compartments and the corresponding comprised target particles, e.g. first and/or second sensor data as provided by the first and/or the second sensor unit, respectively, may be created during and/or after the takeup process, and before and/or during the deposition process. Such data may generally be stored for later use. For example, data created during and/or after the takeup process but before the deposition process may be used in the deposition process. For example, the data may include information about the suitability of each compartment for further processing, i.e. for deposition. Unsuitable compartments may be excluded from deposition in the deposition process, e.g. by selectively inhibiting deposition of the respective compartments from the microchannel of the deposition device.

The method may include that the supporting fluid is immiscible with the sample fluid, preferably wherein the supporting fluid is the carrier fluid.

The present invention also relates to methods that combine takeup and deposition of microscopic objects. Particularly, the present invention relates to a method for takeup and deposition of objects, comprising one of the methods for takeup of target objects as described above and one of the methods for depositing the target objects as described above.

### BRIEF DESCRIPTION OF THE DRAWINGS

The invention will be explained in more detail in the following text with reference to preferred exemplary embodiments which are illustrated in the appended schematic drawings, in which:

FIG. 1 shows an embodiment of a system according to the invention.

FIG. 2 shows another embodiment of a system according to the invention.

FIGS. 3a and 3b show a functional concept according to the invention.

FIGS. 3c and 3d show a functional concept according to the invention.

FIG. 4 shows a takeup process according to the invention.

FIG. 5 shows a takeup process according to the invention.

FIG. 6 shows a concept according to the invention including prearranged microscopic objects.

FIG. 7 shows an embodiment of a deposition process according to the invention.

FIG. 8 shows an embodiment of a deposition process according to the invention.

FIG. 9 shows an embodiment of a deposition process according to the invention.

FIG. 10 shows an embodiment of a microfluidic device according to the invention.

FIG. 11 shows an embodiment of a microfluidic device according to the invention including an optical element.

FIG. 12 shows an embodiment of a takeup process with variable spacing between sample fluid compartments according to the invention.

FIG. 13 shows an embodiment of a takeup process with variable spacing between sample fluid compartments according to the invention.

### DETAILED DESCRIPTION

The figures are schematic drawings and as such may not show all details of the systems and their components. Particularly, the drawings are not to scale and the shown dimensions are only exemplary and may vary. The drawings illustrate exemplary embodiments to provide a thorough understanding of the present invention. The drawings are not intended to limit the scope of the invention, as defined by the appended claims.

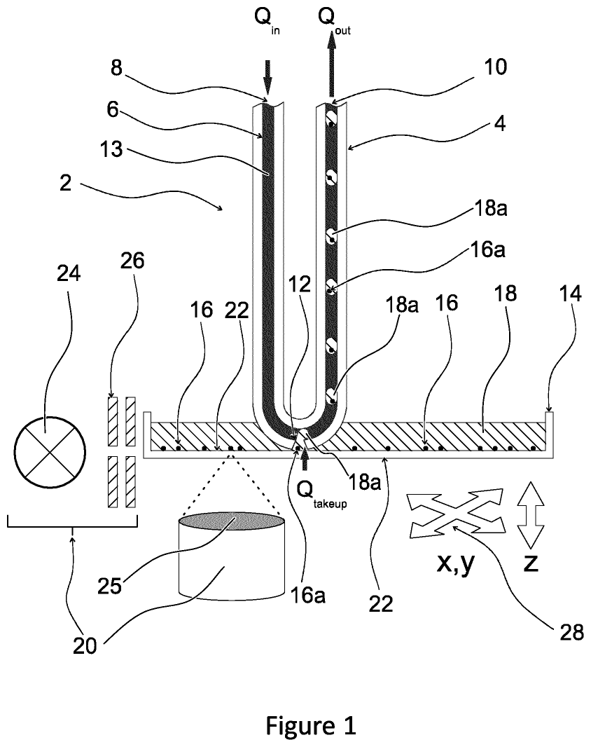

FIG. 1 schematically shows an embodiment of a system 2 for handling microscopic objects, particularly for the take-up of microscopic objects, according to the invention. FIG. 1 shows in cross-section a first microfluidic device 4 defining a microchannel 6 with an inlet 8 and an outlet 10 and an opening 12 between the inlet 8 and the outlet 10. The device 4 may be implemented as a tube, e.g. a polymeric tube, with the opening 12 being preferably located at a loop portion of the tube. The opening 12 may have a cone-like or truncated cone-like shape. However, any other first microfluidic device described herein is suitable.

The system 2 further comprises a conveying device (not shown). The conveying device is configured to pump a carrier fluid 13 via the inlet 8 into the microchannel 6 with an input volumetric flow rate Qᵢₙ and to remove fluid from the microchannel 6 via the outlet 10 with an output volumetric flow rate Qₒᵤₜ.

FIG. 1 further shows (in cross-section) a sample container 14 (also referred to as a “container” 14 herein) for providing a sample comprising dispersed microscopic objects 16 (also referred to as “objects” 16 herein) in a sample fluid 18.

The cross section, i.e. the takeup-diameter, of the opening 12 is selected such that, if the opening 12 is in the sample fluid 18, the input volumetric flow rate Qᵢₙ is equal to the output volumetric flow rate Qₒᵤₜ (i.e. Qᵢₙ=Qₒᵤₜ) no carrier fluid emerges from the opening 12 into the sample fluid 18 and no sample fluid 18 enters the microchannel 6, and, if the output volumetric flow rate Qₒᵤₜ is greater than the input volumetric flow rate Qᵢₙ (i.e. Qₒᵤₜ>Qᵢₙ) sample fluid 18 enters the microchannel 6 via the opening 12 so that it is embedded as one or more compartments of sample fluid 18a in the flow of the carrier fluid 13.

The system 2 is configured for a takeup mode in which, at least intermittently, the output volumetric flow rate Qₒᵤₜ is greater than the input volumetric flow rate Qᵢₙ, i.e. Qₒᵤₜ>Qᵢₙ, so that the system 2 provides a flow of carrier fluid 13 from the inlet 8 through the microchannel 6 and past the opening 12 to the outlet 10 and sequentially takes up one or more compartments of sample fluid 18a into the flow of the carrier fluid 13 with a takeup volumetric flow rate Qₜₐₖₑᵤₚ.

FIG. 1 also shows a first imaging unit 20, i.e. a first sensor unit, for imaging the objects 16 in the container 14. As shown, the objects may sediment to the bottom of the container 14, which may have a suitably shaped surface 22 for imaging and takeup of the objects 16. Preferably the surface 22 is essentially flat. The imaging unit 20 may comprise a light source 24 for illuminating and/or exciting the objects 16 for imaging with the imaging unit 20 and a light collecting means 25 such as a lens or lens system 25, such as an objective, for collecting light, i.e. for imaging the objects 16. As shown, the imaging unit 20 may comprise additional optical elements 26, such as one or more shutters, irises, lenses, reflectors, beam splitters and/or the like, for directing the light emitted by the light source 24 to the objects 16 as required. The light source 24 may be located so as to illuminate the objects 16 from a lateral direction and the light collecting means 25 may be located below the objects 16. The light collecting means 25 may be connected to a camera (not shown) or other detector for creating corresponding sensor/imaging data. The first sensor (here imaging unit 20) may be configured for identifying positions of the dispersed objects in the sample fluid, including a position of a target object 16a selected from the dispersed objects.

As shown in FIG. 1, the system 2 may comprise a positioning unit 28 configured to position the opening 12 at a target position proximate the position of a target object 16a such that the target object 16a is drawn into the microchannel 6 together with a respective compartment of sample fluid 18a and thereby embedded with the sample fluid 18 in the carrier fluid 13. The positioning process may comprise a movement of the opening 12 relative to a reference frame and/or of the target object 16a relative to a reference frame. This means that the positioning unit 28 may be configured to move the first sensor (here imaging unit 20) or parts thereof, the container 14 and/or the first microfluidic device 4 relative to one another. For example, the system 2 may comprise a stage configured for movement in at least two, preferably three dimensions, commonly referred to as a righthanded cartesian XYZ-system, wherein X and Y denote two horizontally oriented directions and Z denotes a vertical direction. The container 14 may be mounted on the stage to be moved by the stage. The positioning unit 28 may (alternatively or additionally) comprise a unit for movement of the first microfluidic device 4 in three dimensions, i.e. in X, Y and Z. The positioning unit 28 may also comprise a unit for movement of the first sensor (imaging unit 20) in three dimensions, i.e. in X, Y and Z. The first sensor may move together with the opening 12.

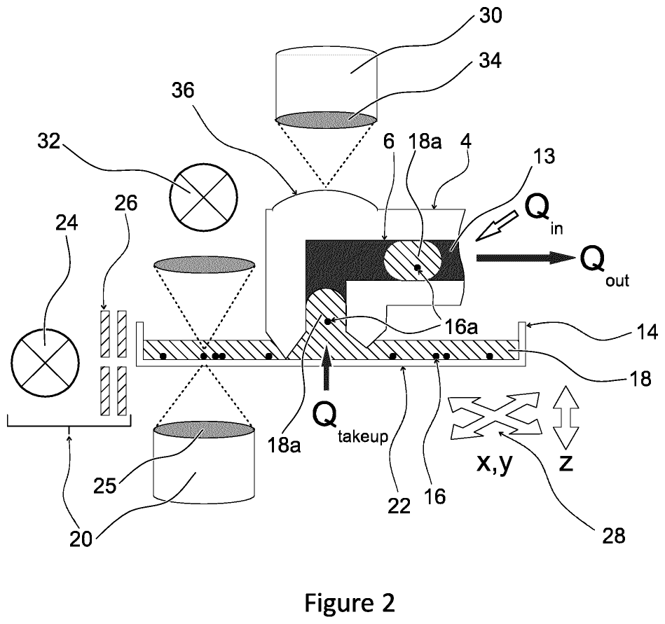

FIG. 2 shows an alternative embodiment of a system 2 according to an aspect of the invention. The system 2 of FIG. 2 comprises all components as described for the system 2 in FIG. 1. In addition, as shown in FIG. 2, the system 2 may comprise a second sensor unit. As shown, the second sensor unit may be a second imaging unit 30. The second imaging unit 30 may comprise a light source 32 and a light collecting means 34. Again, additional optical components (not shown) as described above may be used to control the light path from the light source to the objects 16 and from the objects 16 to the light collecting means 34. The light collecting means 34 of the second imaging unit may be a lens, a lens system, such as an objective, and/or the like. The second sensor (here imaging unit 30) may be arranged similarly to the first sensor (here imaging unit 20) or differently, for example with the light source 32 and the light collecting means 34 above the objects 16. As shown, the second sensor (imaging unit 30) may be arranged such that it is configured to image the sample fluid compartments 18a in the microchannel 6. For example, the light collecting means 34 of the second sensor (imaging unit 30) may be arranged above the first microfluidic device 4 and the light source 32 of the second sensor (imaging unit 30) may be arranged laterally as compared to the microchannel 6.

As also schematically shown in FIG. 2, the light collecting means 25 could be arranged above the container 14.

As shown in FIG. 2, the first microfluidic device may comprise an optical element 36. As shown, the optical element 36 may be configured for coupling light into and/or out of the microchannel 6. In the case of FIG. 2 it is used to couple light out of the microchannel 6 for imaging the sample fluid compartments 18a with the second imaging unit 30. However, as described above, the optical element 36 may be a lens, glass window, or the like and may also be used to couple in a laser, for example for the purposes described above.

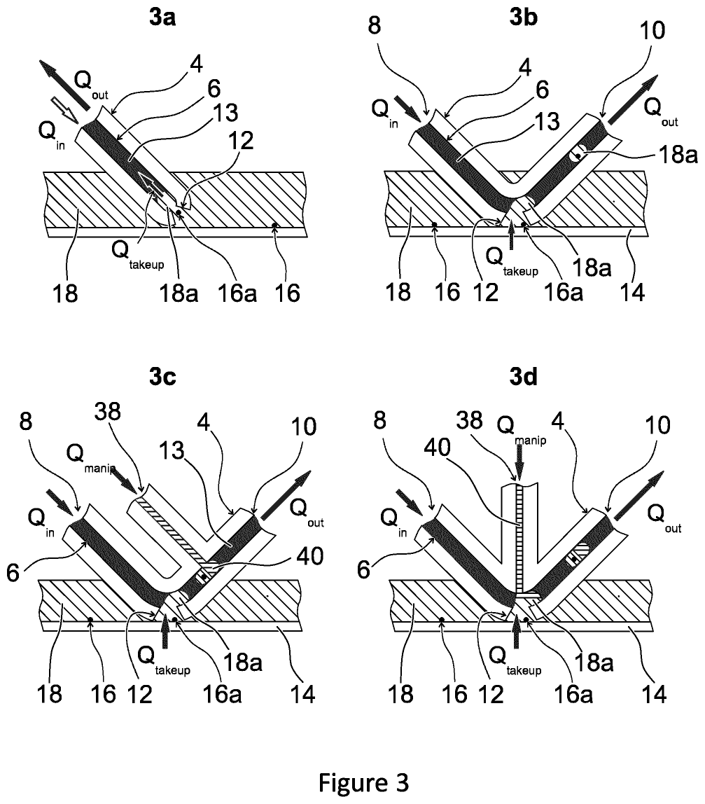

FIGS. 3a and 3b show a functional concept according to the invention. FIG. 3a shows a cross-sectional side view of a first microfluidic device 4, particularly a microchannel 6. FIG. 3b shows the same device in front view. FIGS. 3a and 3b illustrate the balance of volumetric flow rates in a takeup mode of a microchannel 6 with an inlet 8 an outlet 10 and an opening 12. Then Qₒᵤₜ=Qᵢₙ+Qₜₐₖₑᵤₚ. FIGS. 3a and 3b depict the case of |Qₒᵤₜ|>|Qᵢₙ|, which causes a takeup volumetric flow rate Qₜₐₖₑᵤₚ with |Qₜₐₖₑᵤₚ|>0, resulting in the takeup of sample liquid compartments 18a that are embedded in the carrier fluid 13 flowing from the inlet 8 past the opening 12 to the outlet 10 of the microchannel. As a target object 16a is located close to the opening 12, the target particle 16a is drawn into the microchannel 6 together with the sample fluid compartment 18a. In other words, the target particle 16a is embedded in the sample fluid compartment 18a, which is in turn embedded in the carrier liquid 13.

FIG. 3c shows another functional concept according to the invention. FIG. 3c shows a cross-sectional front view of a first microfluidic device 4. In addition to a microchannel 6 with an inlet 8 an outlet 10 and an opening 12, the embodiment of FIG. 3c comprises a manipulation inlet 38 that joins the microchannel 6 between the opening 12 and the outlet 10. FIG. 3c illustrates the balance of volumetric flows in a takeup mode of a microchannel 6 with an inlet 8, an outlet 10, an opening 12 and a manipulation inlet 38. As already noted, the sizes of the channels, inlets and outlets are only schematical and exemplary and may thus vary. For the case shown in FIG. 3c the following equation is valid: Qₒᵤₜ=Qᵢₙ+Qₜₐₖₑᵤₚ+Qₘₐₙᵢₚ. FIG. 3c depicts the case of |Qₒᵤₜ|>|Qᵢₙ+Qₘₐₙᵢₚ|, which causes a takeup volumetric flow rate Qₜₐₖₑᵤₚ with |Qₜₐₖₑᵤₚ|>0 through the opening 12, resulting in the takeup of sample liquid compartments 18a that are embedded in the carrier fluid 13 flowing from the inlet 8 past the opening 12 to the outlet 10 of the microchannel.

As shown, the first microfluidic device may be configured to add a manipulation fluid 40 to a sample fluid compartment 18a upon the compartment passing the manipulation inlet 38. Properties and specific examples for manipulation fluids 40 have already been given above. In general, such a manipulation fluid 40 may be used to manipulate (a) sample fluid compartment(s) 18a, for example in order to change the size of the compartment(s) 18a or in order to provide one or more components to the sample fluid compartment(s) 18a, for example in order to bring these components into contact with the target object(s) 16a comprised in the compartment(s) 18a. For example, if the objects are one or more cells, e.g. plant embryos, one may add one or more substances for manipulating the cell(s)/object(s) and/or for investigating the reaction of the cells to the substance(s). The manipulation fluid may also be a gas that may be metabolized by the cell(s)/object(s).

As in FIG. 3c, FIG. 3d shows again a device 4 with a manipulation inlet 38 and the same volumetric flow conditions, with the difference that the manipulation inlet 38 is located in the vicinity of the opening 12, such that adding of manipulation fluid 40 occurs upon takeup of a sample fluid compartment 18a into the microchannel 6.

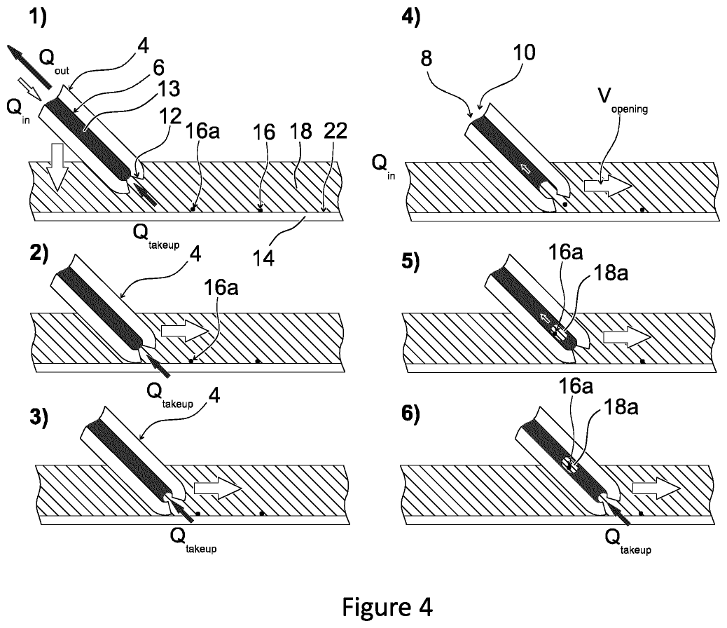

FIG. 4 shows a takeup process, i.e. taking up a target object 16a in crosssectional side view. In FIG. 4-1), a first microfluidic device 4 as described above is positioned in a sample fluid 18 with dispersed microscopic objects 16 being located on a surface 22 of a container 14. In FIG. 4-2), the first microfluidic device 4 is moved relative to a target object 16a to position the opening 12 of the first microfluidic device 4 at a target position close to the target particle 16a. FIG. 4-3) illustrates that during such movement the input and output volumetric flow rates Qᵢₙ, Qₒᵤₜ may be set to have |Qₒᵤₜ|>|Qᵢₙ|, which causes a takeup volumetric flow rate Qₜₐₖₑᵤₚ with |Qₜₐₖₑᵤₚ|>0, resulting in the takeup of sample liquid through the opening 12 into the microchannel 6. Without wanting to be bound by theory, it is assumed that such takeup of sample liquid during movement of the opening may compensate for a movement of sample fluid 18 in the container as effected by the movement. Such movement of sample fluid 18 would otherwise be expected to move the target object 16a such that takeup of the target object 16a would be inhibited. Accordingly, it is preferred that the velocity of the opening v_{opening} is adapted to the the takeup volumetric flow rate Qₜₐₖₑᵤₚ.

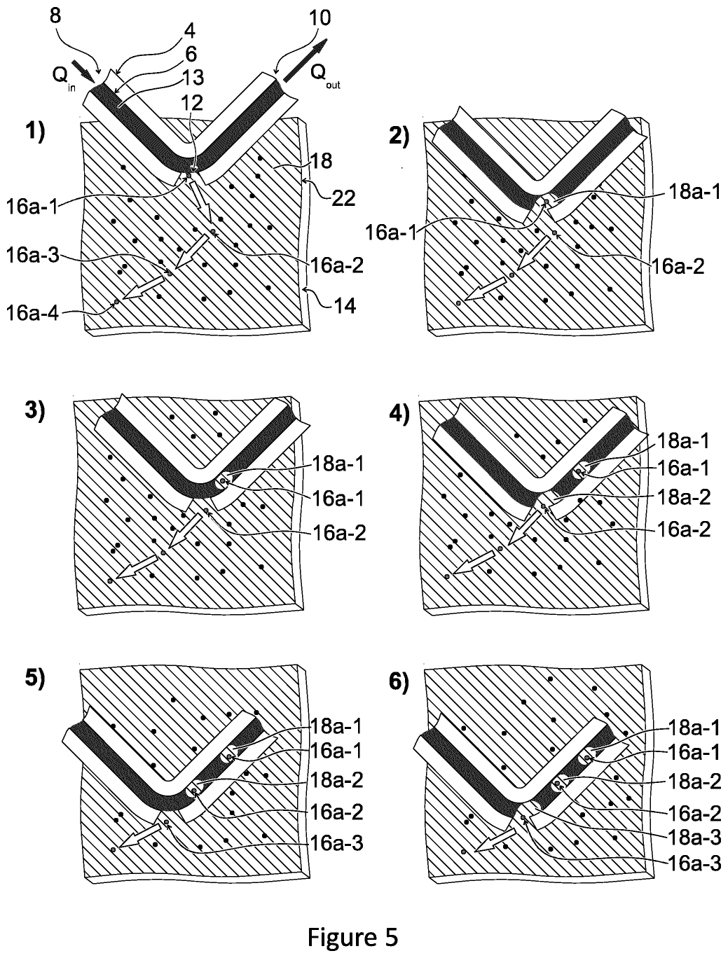

As shown in FIG. 4-4), the opening may arrive at the target position just in time to suck the target particle 16a into the microchannel 6 together with a corresponding sample fluid compartment 18a, thereby embedding the target particle 16a in the sample fluid compartment 18a and the sample fluid compartment 18a in the carrier fluid 13 flowing from the inlet 8 past the opening 12 and to the outlet 10 of the microchannel 6. While the sample fluid compartment is 18a further moves, together with the carrier fluid 13, towards the outlet 10 of the microchannel 6, the first microfluidic device 4 and/or the opening 12 may be further moved relative to another target particle 16a (FIGS. 4-5) and 4-6)) and the steps described with FIG. 4 may be repeated.

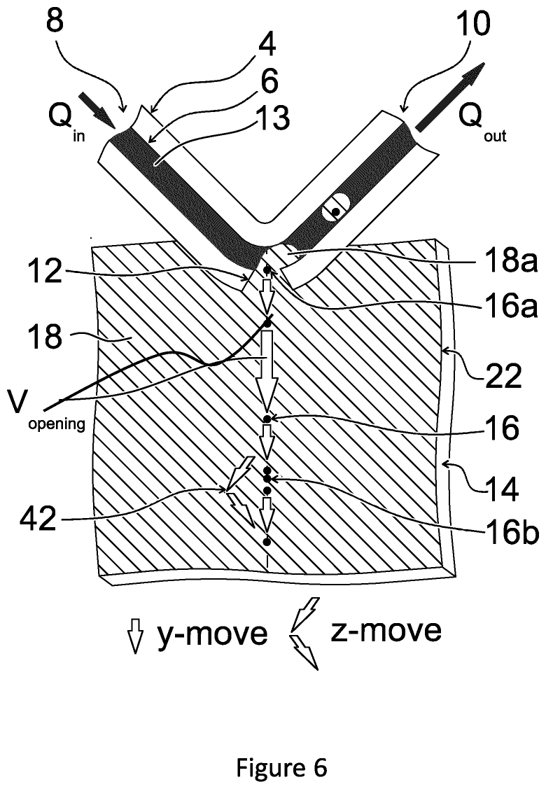

A horizontal movement, i.e. X,Y-movement, of the opening 12 is explained with reference to FIG. 5. FIG. 5-1) shows a first microfluidic device 4 as mentioned above in top view. It comprises an inlet 8 an outlet and an opening 12. In FIG. 5-1) the microchannel 6 only comprises carrier fluid 13 flowing from the inlet 8 to the outlet 10. A first target object 16a-1 is about to be sucked into the microchannel 6 together with a corresponding sample fluid compartment 18a-1. The arrows indicate the path or trajectory in X and Y direction that the opening 12 is going to follow in order to take up the target objects 16a-1 to 16a-4. In FIG. 5-2), the first target object 16a-1 is being compartmentalized, while the opening 12 may already be moving on to the next target object 16a-2. In FIG. 5-3), the first target object 16a-1 is fully compartmentalized, the opening has reached the target position of and is taking up the second target object 16a-2. In FIG. 5-4), the second target object 16a-2 is being compartmentalized and the first target object 16a-1 in the first sample fluid compartment 18a-1 is already located further towards the outlet 10. In FIG. 5-5), the second target object 16a-2 is fully compartmentalized in a second sample fluid compartment 18a-2, the opening 12 has reached the target position of and is taking up the third target object 16a-3. In FIG. 5-6), the third target object 16a-3 is being compartmentalized. During compartmentalization of the target particles, the opening 12 may be moving in order to save time and already approach the next target object 16a, as shown with FIGS. 5-1) to 5-2). Alternatively, the opening may not move during compartmentalization of a target object 16a, as shown with FIGS. 5-3)-5-4) and 5-5) to 5-6). Both options may be combined in a trajectory or path of the opening 12, e.g. in a takeup sequence.

It is noted that as long as the first target position has not been reached (FIG. 5-1)), the volumetric flow rates may be set to have |Qₒᵤₜ|=|Qᵢₙ| to avoid unnecessary takeup of sample fluid 18 into the microchannel 6. However, upon approaching the first target position the volumetric flow rates may be set to have |Qₒᵤₜ|>|Qᵢₙ|, which causes a takeup volumetric flow rate Qₜₐₖₑᵤₚ through the opening 12 with |Qₜₐₖₑᵤₚ|>0, resulting in the takeup of sample fluid 18 into the microchannel 6.

The microscopic objects 16 and/or the target objects 16a may be prearranged, preferably prearranged on a surface. An exemplary embodiment is shown in FIG. 6. As shown, the objects 16 may be prearranged in a row, e.g. by having one or more corresponding depressions in the surface, e.g. a furrow. Upon sedimentation of the objects 16 onto the surface 22 of a container 14, possibly combined with induced lateral movement of the objects 16 relative to the surface 22, e.g. by placing the container with the sample on a shaker, the objects 16 preferably localize into the depressions.

FIG. 6 also shows that the velocity of the opening v_{opening} may be adapted to the distance between consecutive target objects 16a, e.g. in order to have regularly spaced compartments of sample fluid, whith each sample fluid compartment 18a comprising a target object 16a for constant Qₒᵤₜ and Qᵢₙ. If the target objects are located further apart, the velocity v_{opening} is chosen to be higher (long arrows) and if the target objects are located closer together, the velocity v_{opening} is chosen to be lower (short arrows).

In case there are objects 16 that are classified to be unsuitable for take up, such as object cluster 16b, in which the individual objects are too close together for individualized takeup, the opening 12 may move around such unsuitable objects 16. This is indicated by arrows 42 in FIG. 6. This bypass movement may occur in X, Y and/or Z direction, in FIG. 6 a bypass in Z is indicated. The longer path may be compensated by a higher velocity v_{opening} of the opening 12.

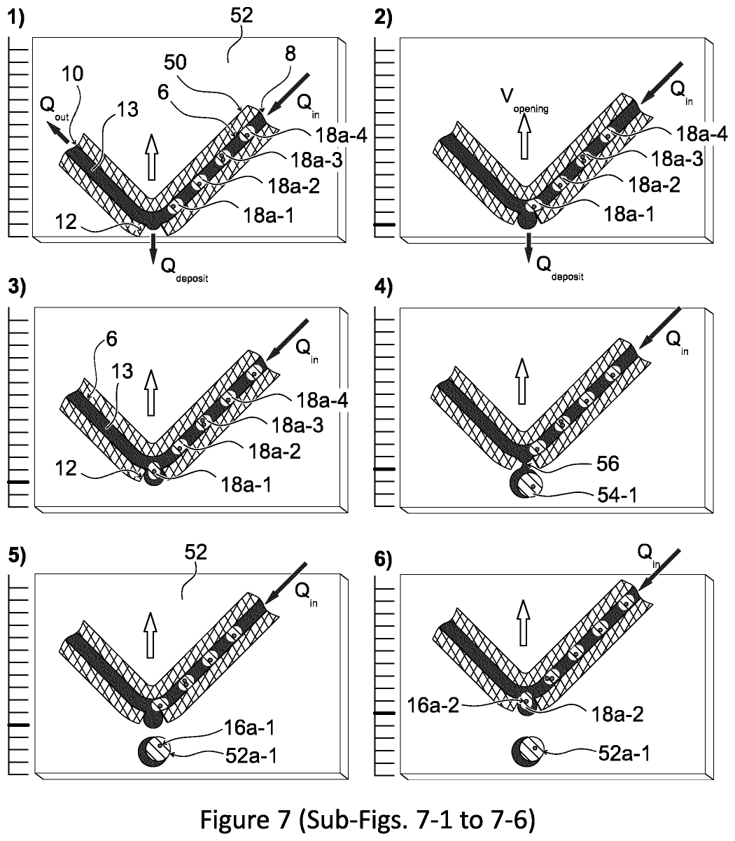

FIG. 7 schematically shows object deposition. As shown, the deposition may occur via a deposition device 50 that is or is like the first microfluidic device 4. The situation is shown in top-view, i.e. the paper plane corresponds to the X-Y-plane. The deposition device 50 (shown in a cross section) comprises a microchannel 6 with an inlet 8, an outlet 10 and an opening 12 between the inlet 8 and the outlet 10. The scale at the left side of each sub-figure has been drawn in order to facilitate the indication of the position of the opening (indicated by thicker line in scales). The opening 12 moves in Y-direction in all sub-figures of FIG. 7, but this is only exemplary.

FIG. 7-1): A sequence of sample fluid compartments 18a-1 to 18a-4 embedded in a flow of carrier fluid 13 is provided in the microchannel 6 via the inlet 8. The compartments each comprise at least one microscopic object. The opening 12 moves in Y-direction in such a distance to a target surface 52 (in Z-direction) that drops emerging from the opening 12 may contact the target surface 52. The target surface is wettable with the sample liquid and less- or non-wettable with the carrier liquid. The target surface is more wettable with the sample liquid than with the carrier liquid. As the volumetric flow rates are set to have |Qₒᵤₜ|<|Qᵢₙ|, a deposition volumetric flow rate Q_{deposit} through the opening 12 with |Q_{deposit}|>0 is caused. This results in fluid exiting the microchannel 6 through the opening 12. As shown in FIG. 7-1), carrier fluid 13 starts to emerge from the opening 12.

FIG. 7-2): further carrier fluid 13 emerges and the first sample fluid compartment 18a-1 is transported to the opening 12, while the opening 12 is moving in Y-direction.

FIG. 7-3): The first sample fluid compartment 18a-1 is emerging from the opening 12 together with a portion of carrier fluid 13, while the other sample-fluid compartments 18a-2 to 18a-4 are being transported further towards the opening 12 with the flow of carrier fluid 13 in the microchannel 6.

FIG. 7-4): The first sample fluid compartment 18a-1 is still further emerging from the opening 12 together with a portion of carrier fluid 13, thus forming a drop 54-1. The drop 54-1 contacts the target surface and a neck 56 in the carrier fluid 13 is emerging, while the opening 12 is further moving in Y-direction.

FIG. 7-5): The opening 12 has further moved in Y-direction and the drop 54-1 has separated from the fluid(s) in the microchannel, i.e. the neck 56 has ruptured, thereby depositing the sample fluid compartment 18a-1 with the target object 16a-1 at a first target site 52a-1 on the target surface 52. The formation of the drop 54-1 and the rupturing of the neck 56 is supported by the target surface 52 being an unfavorable target for the carrier fluid 13 and a favorable target for the sample fluid, i.e. the target surface being non-wettable to the carrier fluid 13 and/or less wettable by the carrier fluid 13 than by the sample fluid 18 and/or wettable by the sample fluid 18. On the other hand, these surface properties support the adhesion of the sample fluid. In addition, the second compartment 18a-2 in the microchannel 6 is approaching the opening 12.

FIGS. 7-6) and 7-7) and 7-8): The processes described in FIGS. 7-3), 7-4) and 7-5) for the first compartment 18a-1) are repeated for the second compartment 18a-2 comprising a second target object 16a-2 to be deposited (drop 54-2) at a second target site 52a-2 on the target surface 52. In addition, the third compartment 18a-3 in the microchannel 6 is approaching the opening 12.

FIG. 7-9): As the third compartment 18a-3 in the microchannel 6 comprises two microscopic objects 16a, the compartment 18a-3 is classified as unsuitable for further processing and deposition of the compartment 18a-3 is inhibited. Instead, the unsuitable compartment 18a-3 is transported past the opening 12 towards the outlet 10. Such classification may generally occur on the flight, i.e. during the deposition process, or may be done in advance. Data for the classification may be collected by the first and/or the second sensor unit. Data for the classification may be collected before, during and/or after the takeup process and/or data for the classification may be collected before and/or during the deposition process. Furthermore, the compartment 18a-4 is approaching the opening 12 in the microchannel 6.

FIG. 7-10): The deposition process is repeated for the next compartment 18a-4 (corresponding to FIG. 7-6)) at a new target site 52a-4. The velocity of the opening v_{opening} may be controlled as required. For instance, v_{opening} may be kept constant. This may result in target sites 52a that are spaced according to v_{opening} and according to the spacing of the sample fluid compartments in the microchannels.

Compartments 18a that are not deposited result in empty potential target sites 52a on the surface, i.e. in gaps in the pattern of drops on the target surface. The velocity v_{opening} may also be adapted to the spacing of the sample fluid compartments 18a in the microchannel 6 that are to be deposited and the suitability of the compartments 18a for deposition. Again, this can be done on the flight or based on previously collected data.

If the deposition process shown in FIGS. 7-1) to 7-10) is done on a liquid absorbing target surface 52, the target sites 52a arise as soaked areas by wicking of the surface touching sample liquid 18 and/or carrier liquid 13. The dispersed objects 16a remain on the surface.

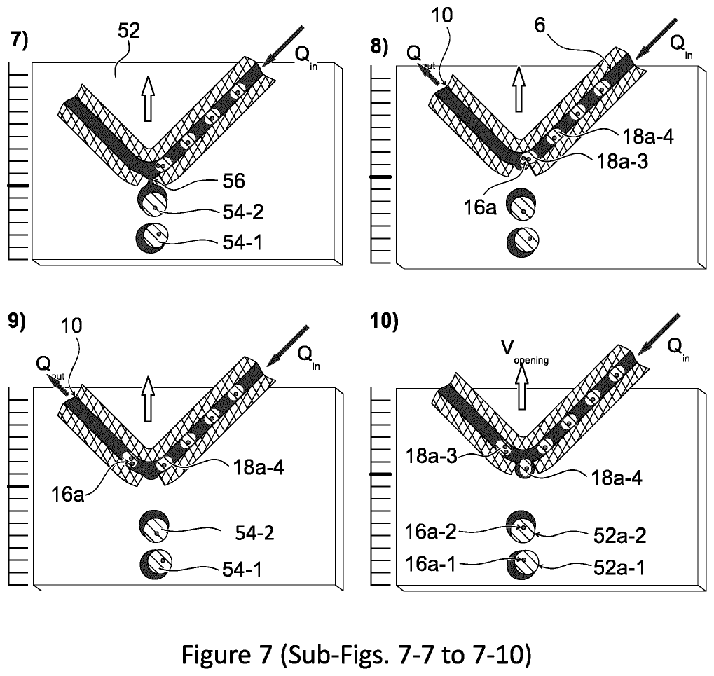

As shown in FIG. 8, the target surface 52 may comprise a, preferably regular, pattern of areas 58 that are wettable by the sample fluid and non-wettable by the carrier fluid in at least one area 60 that is non-wettable by the sample fluid 18 and the carrier fluid 13. The areas 58 represent target sites 52a and the system may be configured to deposit the sample fluid compartments 18a at these areas 58. The movement and particularly the velocity v_{opening} of the opening 12 across the target surface 52 and the volumetric flow rate out of the opening Q_{deposit} may be controlled accordingly.

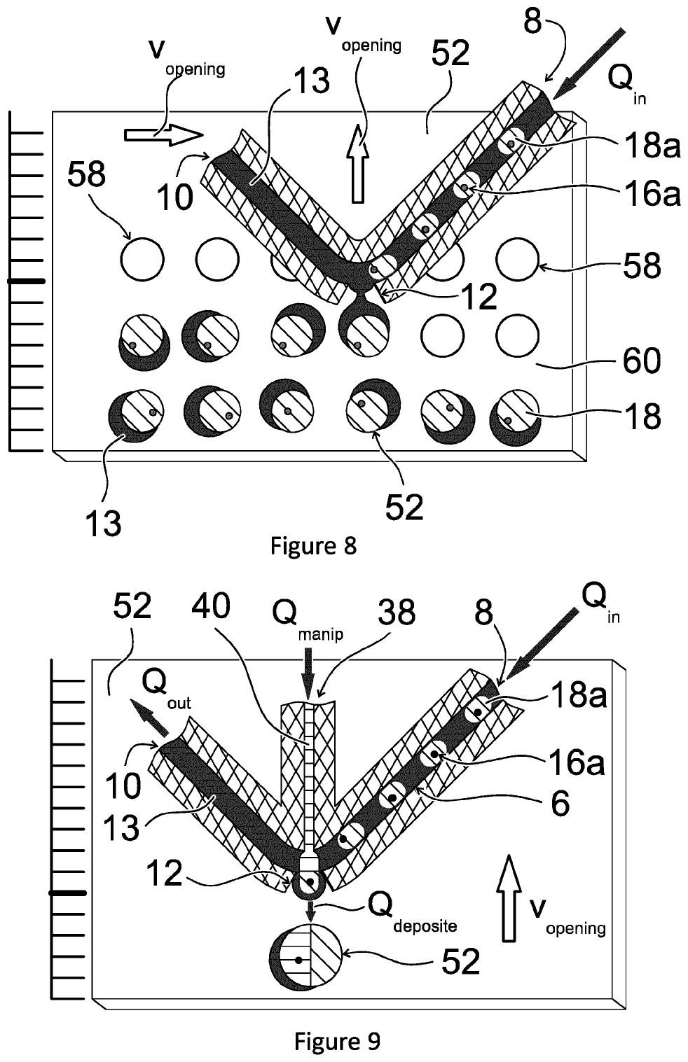

As shown in FIG. 9, the compartments 18a including the target objects 16a may be deposited as described above, with the further feature of providing manipulation fluid 40 to the sample fluid compartments 18a for deposition. The microfluidic device may correspond to the device described with reference to FIG. 3d) above. As shown in FIG. 9, the deposition device may comprise a manipulation inlet 38 that joins the microchannel 6 in the vicinity of the opening 12. For the case shown in FIG. 9 the following equation is valid: Qₒᵤₜ+Q_{deposit}=Qᵢₙ+Qₘₐₙᵢₚ. FIG. 9 depicts the case of |Qₒᵤₜ|<|Qᵢₙ+Qₘₐₙᵢₚ|, which causes a deposition volumetric flow rate Q_{deposit} with |Q_{deposit}|>0 through the opening 12, resulting in the deposition of sample liquid compartments 18a that are embedded in the carrier fluid 13 flowing from the inlet 8 past the opening 12 to the outlet 10 of the microchannel 6. As shown, the deposition device may be configured to add a manipulation fluid 40 to a sample fluid compartment 18a upon the compartment 18a passing the manipulation inlet 38 and exiting the microchannel 6 via the opening 12. Properties, specific examples for manipulation fluids 40 and the purpose of adding manipulation fluid 40 to the compartments 18a have already been given above. In addition, in the deposition process, adding manipulation fluid to the microchannel also contributes to ejecting the compartments 18a through the opening.

The deposition device may also be analogous to the microfluidic device of FIG. 3c. In this case, the manipulation inlet 38 is located between the inlet 8 and the opening 12, such that adding of manipulation fluid 40 occurs before the sample fluid compartments 18a reach the opening 12 of the microchannel 6.

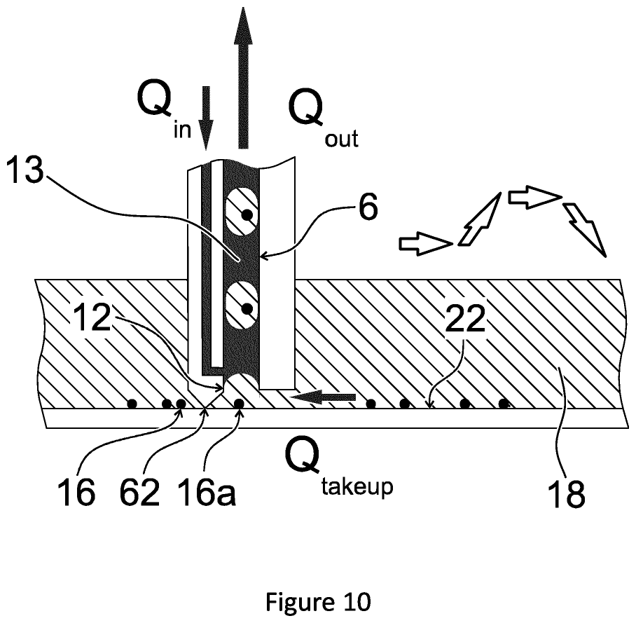

The microfluidic device may have various specific layouts. For example, the layout of a microfluidic device may be such that the portion of the microchannel 6 with the opening 12 crosses a direction of movement of the device during takeup and/or deposition, e.g. essentially runs perpendicular to the direction of movement, as shown in FIGS. 1-9. Alternatively, as shown in FIG. 10, the portion of the microchannel 6 with the opening 12 may run essentially in a plane parallel to the direction of movement. Moreover, the size of the microchannel 6 may vary within a microfluidic device. For example, the microchannel 6 may have first cross section and a second cross section, wherein the second cross section differs from the first cross section, e.g. in size/diameter and/or shape. Particularly, the portion of the microchannel 6 between the inlet 8 and the opening 12 may have a smaller diameter than the portion of the microchannel 6 between the opening 12 and the outlet 10. Such device can be created in different materials, for example glass, polymer and/or silicon, by 3D printing, stereo lithography and/or other micro system engineering technologies. The skilled person will thus appreciate that the opening does not necessarily have to be provided in a loop.

As further apparent from FIG. 10, at least the portions of the microchannel 6 that are immediately proximate to the opening 12 are arranged at an angle of 80° or more, preferably of 90° or more. In FIG. 10, an angle of 90° is shown. To provide such angle, the last part of the portion of the microchannel 6 leading from the inlet to the opening 12 extends along a bend. Otherwise, the portion of the microchannel 6 leading to the opening 12 and the portion of the microchannel 6 leading away from the opening may extend as desired, for example essentially parallel to each other.

As also shown in FIG. 10, a microfluidic device may comprise a tip and/or protrusion 62 that is configured to shield the opening 12 on one or more sides. The tip/protrusion 62 may be configured to cause several effects. The protrusion 62 may help directing the takeup flow of sample fluid in the sample and at the intended target object 16a. Microscopic objects 16 that are not to be taken up but are close to the target object 16a to be taken up may be kept from entering the opening 12 by the protrusion 62, e.g. when the protrusion is positioned between the unwanted object 16 and the opening 12. Moreover, the protrusion 62 may act as a spacer that automatically ensures the correct distance between the opening 12 and the surface 22.

FIG. 10 further illustrates the flows of fluids (filled arrows) and the movement of the device (empty arrows) during a takeup process. However, the features discussed in the context of FIG. 10 may also be present in a deposition device. However, in this case, preferably the portion of the microchannel 6 between the inlet 8 and the opening 12 may have a larger diameter than the portion of the microchannel 6 between the opening 12 and the outlet 10.

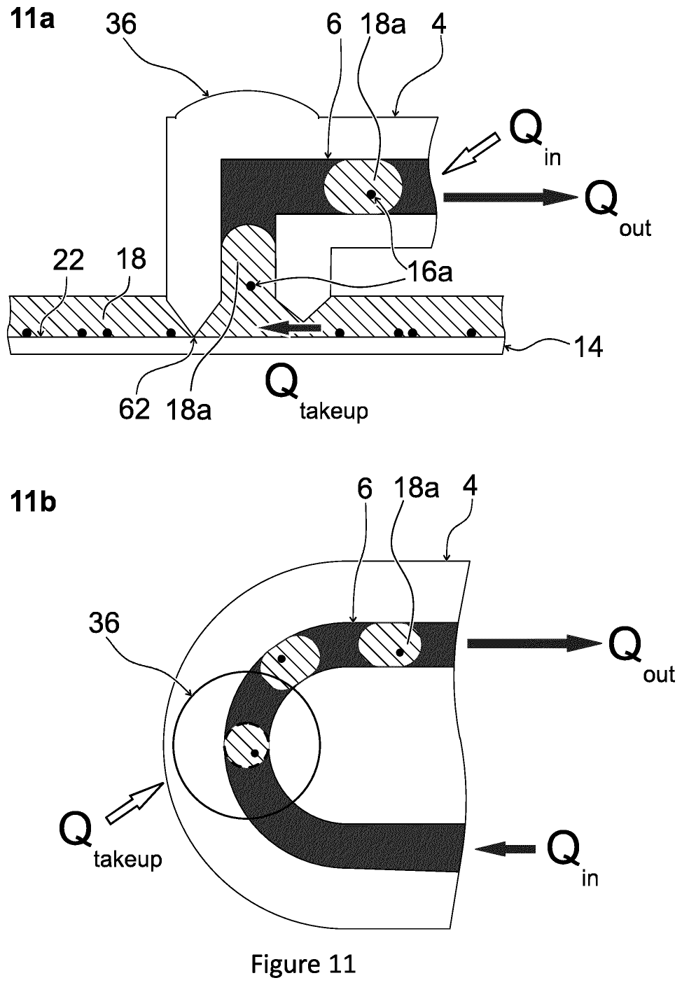

FIG. 11 shows again a takeup scenario with a first microfluidic device 4. As shown, a system and particularly a microfluidic device may combine several of the features already described above. As shown, the microfluidic device may comprise a protrusion 62 and an optical element 36 for coupling in a sensor unit and/or light. FIG. 11a shows a cross sectional side view, FIG. 11b shows a top view. This combination of features is particularly suitable for selective takeup of target particles due to the shielding effect of the protrusion 62 and the option to survey the takeup process, i.e. the nascent sample fluid compartments 18a with the corresponding target objects 16a.

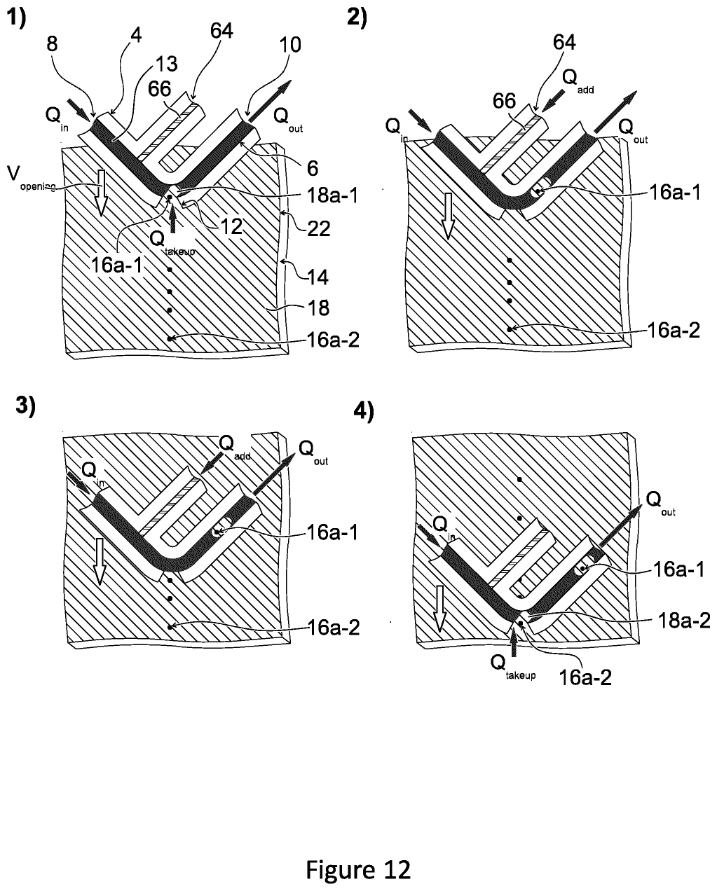

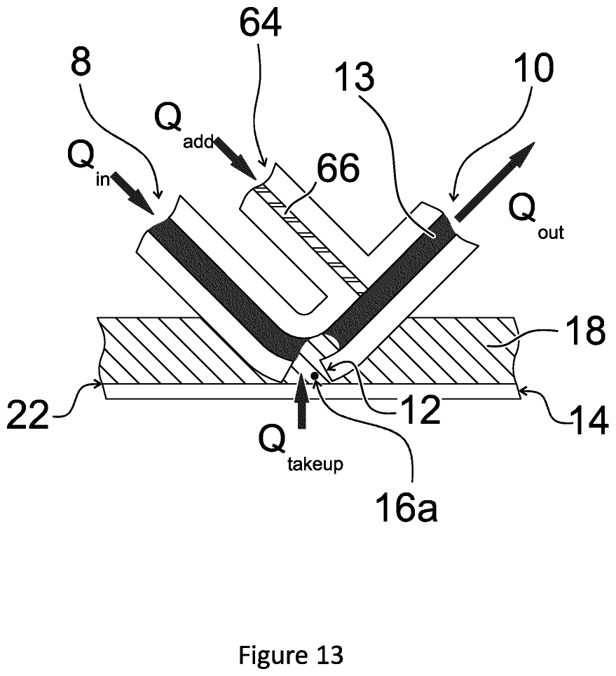

As shown in FIGS. 12, a microfluidic device may comprise an additional inlet 64. Accordingly, the system, the microfluidic device 4 and the additional inlet 64 may be configured to provide additional fluid 66, preferably additional carrier fluid 13, to the microchannel. As shown in FIG. 12, the inlet may be located between the inlet 8 and the opening 12. Alternatively, the additional inlet may be located between the opening 12 and the outlet 10, as shown in FIG. 13. FIG. 12 shows that the pickup or takeup process may be fluidically triggered, e.g. by adding additional fluid 66 to the microchannel 6 in a timed manner, i.e. addition of fluid 66 via the additional inlet 64 is performed whenever takeup of sample fluid 18 from the sample is to be inhibited. In this case, with fixed Qᵢₙ and Qₒᵤₜ, an additional volumetric flow rate of additional fluid Q_{add} is set to Q_{add}=Qₒᵤₜ−Qᵢₙ. When takeup of a sample fluid compartment 18a is desired, the additional volumetric flow rate of additional fluid Q_{add} is set to Q_{add}<Qₒᵤₜ−Qᵢₙ, preferably to zero, in order to allow for takeup volumetric flow. FIG. 12-1) shows the state with Q_{add}=0 and Qₜₐₖₑᵤₚ>0, i.e. a sample fluid compartment 18a-1 with a target object 16a-1 is being created. In FIG. 12-2) the opening 12 is moving on to the next target object 16a-2 as indicated by the empty arrow. Now, Q_{add}=Qₒᵤₜ−Qᵢₙ and Qₜₐₖₑᵤₚ=0. As Qᵢₙ and Qₒᵤₜ are fixed, the sample fluid compartment 18a-1 in the microchannel 6 is transported towards the outlet 10 but takeup of sample fluid is intermittently inhibited. In FIGS. 12-3) and 12-4) the situation is again as in FIGS. 12-1) and 12-2), respectively, but for the next sample fluid compartment 18a-2 with the next target object 16a-2. Generally, Q_{add} may be a function of time, i.e. Q_{add}(t), that may be adapted to the velocity of the opening v_{opening} and the locations of the target objects 16a and/or their distances.

The concept of having a time dependent additional volumetric flow rate Q_{add}(t) to the microchannel 6 may also be adapted to the deposition process. In this case, the deposition device comprises an additional inlet 64 as described above. The time dependent additional volumetric flow rate Q_{add}(t) is then adapted to the desired deposition.

Although specific examples with specific combinations of features were given, the invention is not restricted to these examples. Any combination of features is contemplated.

The following are preferred aspects of the invention:
1. A system for handling dispersed microscopic objects contained in a sample fluid, comprising:
   a first microfluidic device comprising a microchannel with an inlet, an outlet and an opening, the opening being located between the inlet and the outlet;
   a conveying device configured to pump a carrier fluid via the inlet into the microchannel with an input volumetric flow rate Qᵢₙ and to remove fluid from the microchannel via the outlet with an output volumetric flow rate Qₒᵤₜ;
   wherein the cross section of the opening is selected such that, if the opening is in the sample fluid, the following conditions are fulfilled:
      a) if the input volumetric flow rate Qᵢₙ is equal to the output volumetric flow rate Qₒᵤₜ, i.e. Qᵢₙ=Qₒᵤₜ, no carrier fluid emerges from the opening into the sample fluid and no sample fluid enters the microchannel,
      b) if the output volumetric flow rate Qₒᵤₜ is greater than the input volumetric flow rate Qᵢₙ, i.e. Qₒᵤₜ>Qᵢₙ, sample fluid enters the microchannel via the opening so that it is embedded as one or more compartments of sample fluid in the flow of the carrier fluid;
      wherein the system is configured for a takeup mode in which, at least intermittently, the output volumetric flow rate Qₒᵤₜ is greater than the input volumetric flow rate Qᵢₙ, i.e. Qₒᵤₜ>Qᵢₙ, so that the system provides a flow of carrier fluid from the inlet through the microchannel and past the opening to the outlet and sequentially takes up one or more compartments of sample fluid into the flow of the carrier fluid with a takeup volumetric flow rate Qₜₐₖₑᵤₚ;
   wherein the system further comprises:
   a first sensor unit for identifying positions of the dispersed objects in the sample fluid, including a position of a target object selected from the dispersed objects; and
   a positioning unit configured to position the opening at a target position proximate the position of the target object such that the target object is drawn into the microchannel together with a certain volume of sample fluid and thereby embedded with the sample fluid in the carrier fluid as a compartment.
2. The system according to aspect 1, wherein the microscopic objects are dispersed across a support surface, preferably wherein the support surface is the bottom surface of a container in which the sample fluid is contained, the system optionally comprising the support surface and/or the container.
3. The system according to aspect 1 or 2, wherein the first sensor unit is configured for generating corresponding sensor data.
4. The system according to any one of the preceding aspects, wherein the first sensor unit comprises a first imaging unit configured for imaging the dispersed objects and preferably for creating corresponding imaging data.
5. The system according to any of the preceding aspects, comprising a second sensor unit configured for analyzing the target object while the target object is embedded in the compartment of sample fluid embedded in the carrier fluid in the microchannel and preferably for generating corresponding sensor data.
6. The system according to the preceding aspect, wherein the second sensor unit comprises a second imaging unit configured for imaging the target object while the target object is embedded in the compartment of sample fluid embedded in the carrier fluid in the microchannel and preferably for creating corresponding imaging data.
7. The system according to one of the two preceding aspects, wherein the first microfluidic device comprises at least one optical element for the second sensor unit, preferably a lens, a glass window, aperture, refractive element, optical lattice or wave guide, optionally wherein the second imaging unit is provided proximate the opening.
8. The system according to any one of the preceding aspects, wherein the microchannel comprises an additional inlet, wherein the system is configured to deliver a fluid, preferably carrier fluid, into the microchannel via the additional inlet at an additional volumetric flow rate Q_{add}, optionally wherein the additional fluid is selected from: a gas to be metabolized by the objects, a liquid containing a growth stimulator for the objects, and a liquid containing one or more type of bacteria.
9. The system according to the preceding aspect, wherein the system is configured to deliver the fluid via the additional inlet intermittently into the microchannel at determined time points.
10. The system according to any one of the preceding aspects, wherein the microchannel comprises a manipulation inlet arranged at the opening or between the opening and the outlet, and wherein the system is configured to deliver a manipulation fluid via the manipulation inlet into a respective compartment in the microchannel at a manipulation volumetric flow rate Qₘₐₙᵢₚ wherein the fluid delivered via the manipulation inlet is optionally selected from: a gas to be metabolized by the objects, a liquid containing a growth stimulator for the objects, and a liquid containing one or more type of bacteria, a liquid containing culture media composed of nutrition and/or pH-Buffer components, a liquid containing an enzyme, a liquid containing ingredients for digestion of the object, parts of the object or liberation of surface bound molecules, a liquid containing compounds for analytical assay purposes, a liquid containing an PCR mix, a liquid containing antibodies, a liquid containing nanomaterials preferably for surface-enhanced Raman spectroscopy, a liquid containing particles, and/or a liquid containing marker substances preferably dyes.
11. The system according to any one of the preceding aspects, wherein the first microfluidic device comprises a tube, the tube defining the microchannel and the opening, such as a flexible tube made from a polymeric material, preferably from polyamide, polypropylene, polyethylene, polyvinylchloride, polysiloxanes and/or fluorinated polymers such as polytetrafluorethylene (PTFE), perfluorethylenpropylen (FEP), and/or Perfluoroalkoxyalkane (PFA).
12. The system according to the preceding aspect, wherein the tube comprises a loop section and the opening is located at an outer circumference of the loop section, the loop section preferably having a radius of less than 5 mm, preferably less than 1 mm and/or wherein the position of the opening in the tube is oriented towards a target object, when the loop/opening is moved towards the target object.
13. The system according to any one of aspects 1-10, wherein the first microfluidic device comprises a microfluidic chip, preferably a microfluidic glass and/or polymer chip, the microfluidic chip defining the microchannel and the opening.
14. The system according to any one of the preceding aspects, comprising a container in which the sample fluid is contained.
15. The system according to any one of aspects 2-14, wherein the container and/or the support surface comprises a structure for prearranging the microscopic objects, preferably a depression or a pattern of depressions, for example one or more elongated grooves and/or one or more wells.
16. The system according to any one of the preceding aspects, further comprising a tip that is positioned proximate to and/or at least partially around the opening, wherein the tip is configured for contacting a support surface on which the objects are dispersed in order to separate the target object from other dispersed objects and/or to avoid other dispersed objects than the target object from being drawn into the microchannel together with the respective compartment of sample fluid, preferably wherein the tip is tapered and/or terminates at an edge.
17. The system according to any one of the preceding aspects, configured to take up several/a plurality of target objects in a corresponding number of sample fluid compartments.
18. The system according to the preceding aspect, wherein the system is configured to take up the several target objects in immediately consecutive sample fluid compartments immediately consecutively embedded in the carrier fluid in the microchannel.
19. The system according to any one of the preceding aspects, the system comprising a storage configured to accommodate the target object(s) while embedded with the sample fluid in the carrier fluid for storage, cultivation and/or further treatment of the target object(s).
20. The system according to the preceding aspect, wherein the storage comprises, preferably is, a microchannel, such as a tube, and/or a chip device with meandered microchannel.
21. The system according to one of the two preceding aspects, comprising a microfluidic treatment unit.
22. The system according to any one of the preceding aspects, the system being configured to deposit one or more microscopic/target objects embedded with the sample fluid in the carrier fluid at one or more corresponding target sites.
23. The system according to the preceding aspect, wherein the system comprises a microfluidic deposition device for depositing the one or more microscopic/target objects at the target sites, wherein the deposition device comprises a microchannel with an opening and at least one inlet, wherein a plurality of compartments of sample fluid embedded in a carrier fluid are flown via the inlet to the opening, wherein at least some of the compartments of sample fluid are dispensed through the opening and deposited on a target surface.
24. The system according to any of the two preceding aspects, wherein the microchannel of the deposition device is further provided with an outlet, wherein a volumetric flow rate through the inlet of the deposition device when dispensing a compartment Qᵢₙ is greater than a volumetric flow rate through the outlet of the deposition device Qₒᵤₜ when dispensing the compartment of sample fluid, optionally wherein flow through the outlet of the deposition device is blocked or reduced for dispensing the compartment of sample fluid.
25. The system according to any of the three preceding aspects, wherein the microchannel of the deposition device is further provided with a manipulation inlet, wherein the system is configured to deliver a support fluid via the manipulation inlet of the deposition device into the microchannel of the deposition device, preferably at determined time points, to squeeze a compartment of sample fluid located proximate to the opening of the deposition device out of the microchannel of the deposition device through the opening.
26. The system according to the preceding aspect, wherein the manipulation inlet of the deposition device is arranged at the opening of the deposition device, between the inlet and the opening of the deposition device, or between the opening and an outlet of the microchannel of the deposition device.
27. The system according to any of the five preceding aspects,
   wherein the deposition device is provided by a second microfluidic device, preferably a second microfluidic device that is microfluidically connected to the first microfluidic device; or
   wherein the deposition device is provided by the first microfluidic device.
28. The system according to any one of aspects 22-27, comprising a target surface for depositing the object(s).
29. The system of the preceding aspect, wherein the target surface comprises areas that are more wettable by the sample fluid than by the carrier fluid.
30. The system according to one of the two preceding aspects, wherein the target surface comprises a pattern of areas wettable by the sample fluid and one or more areas non-wettable by the sample fluid; and wherein the target sites correspond to the wettable areas.
31. The system according to the preceding aspect, wherein the wettable areas are wettable by the sample fluid and the non-wettable areas are less wettable by the sample fluid, preferably wherein the wettable areas are less wettable by the carrier fluid and/or a support fluid than by the sample fluid, more preferably wherein the non-wettable areas are not wettable by the carrier fluid and/or the support fluid.
32. The system according to one of the two preceding aspects, wherein the wettable areas are patches that are, preferably regularly, arranged on the otherwise non-wettable target surface.
33. The system according to any one of aspects 28-32, wherein the system is configured to change a relative position of the opening of the deposition device to the target surface, preferably via the positioning unit.
34. The system according to the previous, wherein the system is configured to change the relative position of the opening of the deposition device to the target surface continuously, preferably at a constant velocity, and more preferably at a velocity adapted to a pattern of areas wettable by the sample fluid, even more preferably wherein the volumetric flow rate through the inlet when dispensing a compartment Qᵢₙ is constant.
35. The system according to any of aspects 28 to 34, wherein the target surface comprises areas, preferably target sites, that are more absorbent for the sample fluid than for the carrier fluid or vice versa, wherein optionally the absorbent areas correspond to the wettable areas.
36. The system according to any of aspects 28 to 35, wherein target surface may comprise areas, preferably target sites, that are absorbent for both the sample fluid and the carrier fluid, wherein optionally the absorbent areas correspond to the wettable areas.
37. The system according to any of aspects 28 to 36, wherein the target surface comprises a pattern of areas absorbing sample fluid and one or more areas not absorbing sample fluid, and wherein the target sites correspond to areas absorbing sample fluid.
38. The system according to any one of the preceding aspects, wherein the system comprises a control unit configured to control the conveying device, preferably in order to vary the ratio between the input volumetric flow rate Qᵢₙ and the output volumetric flow rate Qₒᵤₜ, and/or optionally to control the delivery of the support fluid via the manipulation inlet.
39. The system according to any one of the preceding aspects, wherein the system comprises the control unit, wherein the control unit is configured to, preferably automatically, control movement of the first and/or second microfluidic devices, and/or takeup, storage, culturing, treatment and/or deposition of the one or more objects.
40. The system according to one of the two preceding aspects, wherein the control unit is configured to identify and/or appraise one or more objects, preferably one or more target objects, among the dispersed objects from the first sensor data created by the first sensor unit, preferably from the first imaging data created by the first imaging unit.
41. The system according to one of the three preceding aspects, wherein the control unit is configured to employ an object recognition algorithm, for example an artificial intelligence object recognition algorithm.
42. The system according to one of the four preceding aspects, wherein the control unit is configured to determine and/or execute a takeup sequence automatically.
43. The system according to any one of the preceding aspects, the system being configured to move the opening at a velocity v_{opening} towards the target object, wherein the velocity v_{opening} is selected such that the takeup volumetric flow rate Qₜₐₖₑᵤₚ compensates or substantially compensates for a displacement of sample fluid towards the target object caused by moving the first microfluidic device through the sample fluid.
44. The system according to any one of the preceding aspects, wherein, at least intermittently, the takeup mode comprises:
   the control unit is configured to keep the takeup volumetric flow rate Qₜₐₖₑᵤₚ constant and the velocity at which the opening is moved v_{opening} between a plurality of consecutive target objects is adjusted according to a distance between each pair of consecutive target objects, preferably wherein the plurality of consecutive target objects are sequentially drawn into the microchannel with respective, equally spaced compartments of sample fluid.
45. The system according to any one of the preceding aspects, wherein, at least intermittently, the takeup mode comprises:
   the takeup volumetric flow rate Qₜₐₖₑᵤₚ is kept constant, the velocity of the opening v_{opening} is kept constant, and a travel path of the opening between a pair of consecutive target objects is adapted accordingly to a distance between the pair of target objects, preferably wherein the pair of target objects are sequentially drawn into the microchannel with respective, consecutive, equally spaced compartments of sample fluid.
46. The system according to any one of the preceding aspects, wherein the microchannel comprises an additional inlet for additionally providing a fluid, preferably a liquid and more preferably the carrier fluid or another carrier fluid that is immiscible with the sample fluid, via the additional inlet to the microchannel at an additional volumetric flow rate Q_{add};
   wherein the takeup mode comprises, at least intermittently:
   the input volumetric flow rate Qᵢₙ and the output volumetric flow rate Qₒᵤₜ are kept constant;
   the additional volumetric flow rate Q_{add} is kept smaller than the absolute value of the difference |Qᵢₙ−Qₒᵤₜ| of the input volumetric flow rate Qᵢₙ and the output volumetric flow rate Qₒᵤₜ, preferably zero, for the takeup of target objects, and preferably also when approaching the opening to the target objects, to allow for takeup of sample fluid through the opening of the first microfluidic device; and wherein otherwise the additional volumetric flow rate Q_{add} is set to be essentially equal to the absolute value of the difference |Qᵢₙ−Qₒᵤₜ| of the input volumetric flow rate Qᵢₙ and the output volumetric flow rate Qₒᵤₜ to prevent takeup of sample fluid through the opening.
47. The system according to any one of the preceding aspects, wherein the takeup mode comprises:
   keeping the output volumetric flow rate Qₒᵤₜ constant and intermittently varying the input volumetric flow rate Qᵢₙ in order to prevent the formation of one or more compartments of sample fluid when the opening is not proximate to a particle or one or more particles proximate to the opening are not to be drawn into the microchannel.
48. The system according to the preceding aspect, wherein the input volumetric flow rate Qᵢₙ is varied by a rapid action valve.
49. The system according to any one of the preceding aspects, further comprising an empty compartment identification means for automatically identifying compartments of sample fluid in which no object is contained.
50. Method for picking microscopic objects by suction, comprising the steps:
   a) providing a sample of dispersed microscopic objects contained in a sample fluid;
   b) providing a first microfluidic device comprising a microchannel with an inlet, an outlet and an opening located between the inlet and the outlet;
   c) providing a carrier fluid that is immiscible with the sample fluid via the inlet to the microchannel at an input volumetric flow rate Qᵢₙ and removing fluid from the microchannel via the outlet at an output volumetric flow rate Qₒᵤₜ;
   d) positioning the opening in the sample fluid;
   e) setting the input volumetric flow rate Qᵢₙ to be smaller than the output volumetric flow rate Qₒᵤₜ, the difference resulting in a takeup volumetric flow rate Qₜₐₖₑᵤₚ of sample fluid through the opening that results in compartments of sample fluid embedded in the carrier fluid;
   f) changing a position of the opening relative to a target object from a starting relative position to a target position to bring the opening proximate to the target object;
   g) drawing the target object into the microchannel together with a respective compartment of sample fluid when embedding the compartment of sample fluid in the carrier fluid according to step e).
51. The method of the preceding aspect, wherein
   a velocity v_{opening} at which the position of the opening relative to a target object is changed in step f) is selected such that the takeup volumetric flow rate Qₜₐₖₑᵤₚ compensates or substantially compensates for a displacement of sample fluid towards the target object caused by moving the first microfluidic device through the sample fluid.
52. The method of one of the two preceding aspects, wherein steps f) and g) are performed for a first target object and repeated for a second target object while keeping the takeup volumetric flow rate Qₜₐₖₑᵤₚ constant, wherein the velocity of the opening v_{opening} is adjusted according to the distance between the first and second target objects to embed the first and second target objects with consecutive compartments of sample fluid, preferably with immediately consecutive and equally spaced first and second compartments of sample fluid, respectively.
53. The method of one of the three preceding aspects, wherein steps f) and g) are repeated while keeping the takeup volumetric flow rate Qₜₐₖₑᵤₚ constant, wherein the velocity of the opening (v_{opening}) is adjusted accordingly for each pair of consecutive target objects to sequentially embed several target objects with a corresponding number of compartments of sample fluid, preferably with a corresponding number of consecutive, equally spaced compartments of sample fluid, more preferably with a corresponding number of immediately consecutive, equally spaced compartments of sample fluid.
54. The method of one of the four preceding aspects, wherein steps f) and g) are performed for a first target object and repeated for a second target object while keeping the takeup volumetric flow rate Qₜₐₖₑᵤₚ constant, wherein the travel path of the opening between the first and second target objects is adapted to embed the first and second target objects with consecutive compartments of sample fluid, preferably with immediately consecutive first and second compartments, respectively.
55. The method according to the preceding aspect, wherein the velocity of the opening v_{opening} is kept constant.
56. The method according to one of the two preceding aspects, wherein the travel path between a pair of immediately consecutive target objects is not straight, preferably wherein the travel path deviates from a straight line parallel to a support surface in lateral directions parallel to the support surface and/or upwards and downwards.
57. The method of any one of aspects 50 to 56, wherein steps f) and g) are repeated for several target objects while keeping the takeup volumetric flow rate Qₜₐₖₑᵤₚ constant, wherein the velocity of the opening v_{opening} is kept constant and wherein the travel path of the opening between the first and second target objects of each pair of consecutive target objects is adapted to sequentially embed several target objects with a corresponding number of compartments of sample fluid, preferably with a corresponding number of consecutive, equally spaced compartments of sample fluid.
58. The method of any one of aspects 50 to 57,
   wherein the microchannel comprises an additional inlet for additionally providing a fluid that is immiscible with the sample fluid via the additional inlet to the microchannel at an additional volumetric flow rate Q_{add}, preferably carrier fluid;
   wherein the input volumetric flow rate Qᵢₙ and the output volumetric flow rate Qₒᵤₜ are kept constant;
   wherein the additional volumetric flow rate Q_{add} is set to be smaller than the absolute value of the difference |Qᵢₙ−Qₒᵤₜ| of the input volumetric flow rate Qᵢₙ and the output volumetric flow rate Qₒᵤₜ, preferably zero, for step g), and preferably for arrival of the opening at the target position in step f), to allow for takeup of sample fluid through the opening; and wherein otherwise the additional volumetric flow rate Q_{add} is set to be essentially equal to the absolute value of the difference |Qᵢₙ−Qₒᵤₜ| of the input volumetric flow rate Qᵢₙ and the output volumetric flow rate Qₒᵤₜ to prevent takeup of sample fluid through the opening.
59. The method of any one of aspects 50 to 58, further comprising the steps:
   analyzing at least some or each compartment of sample fluid, preferably optically, and comparing the, preferably optical, analysis result with a predefined standard, thus creating a labeling result for each compartment;
   labeling each compartment according to the labeling result.
60. The method of any one of aspects 50 to 59, including: providing the sample in a container, wherein the objects are located on a surface of the container, preferably on a bottom surface of the container.
61. The method of any one of aspects 50 to 60, wherein the microchannel further comprises a manipulation inlet arranged at the opening or between the opening and the outlet;
   the method further comprising the step:
      delivering a manipulation fluid via the manipulation inlet into a compartment in the microchannel at a manipulation volumetric flow rate Qₘₐₙᵢₚ;
   preferably wherein Qₒᵤₜ=Qᵢₙ+Qₜₐₖₑᵤₚ+Qₘₐₙᵢₚ+Q_{add}; and/or
   preferably wherein the sample fluid is a liquid and the manipulation fluid is selected from at least one of: a liquid miscible with the sample liquid (e.g., a liquid containing a growth stimulator for the objects, or a liquid containing one or more type of bacteria) and a gas to be metabolized by the objects.
62. The method of any one of aspects 50 to 61, comprising the step:
   prearranging the objects in a specific pattern for takeup, e.g. in one or more lines.
63. The method of any one of aspects 50 to 62, comprising:
   detecting positions of at least some of the objects in the sample, preferably optically;
   selecting the target objects(s) among the objects for which the position was detected.
64. The method of the preceding aspect, wherein the step of selecting the target object(s) comprises:
   identifying and excluding objects that are unsuitable.
65. The method of the preceding aspect, wherein the step of selecting the target object(s) comprises:
   identifying and excluding groups of objects in which the objects are determined to be too close to each other, such as clusters or aggregates of objects;
   preferably wherein groups of objects in which the objects are determined to be too close to each other for embedding them individually into respective compartments of sample fluid are excluded.
66. The method of one of the three preceding aspects, wherein identifying objects, excluding objects and/or selecting the target objects(s) comprises using artificial intelligence.
67. A method for depositing a sequence of microscopic objects that are embedded with a sequence of compartments of a sample fluid in a carrier fluid, the carrier fluid being immiscible with the sample fluid, the method comprising the steps:
   i) providing
      a target surface that is wettable with the sample fluid and/or absorbent for the sample fluid, preferably wherein the surface is absorbent for the carrier fluid, or preferably wherein the surface is poorly wettable with and/or poorly absorbent for the carrier fluid; or
      a target surface that is absorbent for the carrier fluid but poorly absorbent for the sample fluid; or
      a target surface that is less absorbent for the sample fluid than for the carrier fluid; or
      a target surface that is wettable with the sample fluid and/or absorbent for the sample fluid and wherein the surface is less wettable with and/or less absorbent for the carrier fluid;
   j) providing a microchannel having an inlet and an opening through which the objects are dispensed;
   k) providing the sequence of microscopic objects embedded with the sequence of compartments of the sample fluid in the carrier fluid in the microchannel;
   l) moving the opening relative to the target surface, thereby positioning the opening at a target position relative to a target site on the target surface, the target position allowing for drops emerging from the opening to contact the target surface when still in contact with a wall of the microchannel and/or the fluid in the microchannel;
   m) flowing the sequence of compartments through the microchannel towards the opening and dispensing the compartment closest to the opening in the direction of flow through the opening at the target position;
   n) repeating steps I) and m) in order to deposit microscopic objects embedded with different compartments of sample fluid at different target sites.
68. The method of the preceding aspect, wherein the microchannel further comprises an outlet and the opening is located between the inlet and said outlet, the method further comprising the following steps:
   providing fluid via the inlet to the microchannel at an input volumetric flow rate Qᵢₙ and simultaneously removing fluid from the microchannel via the outlet at an output volumetric flow rate Qₒᵤₜ;
   wherein the input volumetric flow rate Qᵢₙ is larger than the output volumetric flow rate Qₒᵤₜ, the difference resulting in a discharge volumetric flow rate Q_{deposit} of sample fluid through the opening that results in deposition of the compartments of sample fluid embedded in the carrier fluid at the target site.
69. The method of one of the two preceding aspects, wherein the repeated positioning of the opening is performed by a continuous movement of the opening, preferably at a constant velocity.
70. The method of one of the three preceding aspects, wherein the target surface comprises a pattern of areas wettable with and/or absorbent for the sample fluid and areas non-wettable with and/or absorbent for the sample fluid; and wherein the target sites correspond to the areas wettable with/absorbent for the sample fluid.
71. The method of the preceding aspect, wherein the repeated positioning of the opening is a continuous movement of the opening, preferably at a constant velocity, and preferably adapted to the pattern of wettable/absorbent and non-wettable/non-absorbent areas of the target surface.
72. The method of one of the two preceding aspects, wherein the wettable/absorbent areas are patches that are, preferably regularly, arranged on the otherwise non-wettable/non-absorbent target surface, and/or
   wherein the areas that are wettable with/absorbent for the sample fluid are absorbent for the carrier fluid.
73. The method of any one of aspects 67 to 72, wherein the microchannel further comprises a manipulation inlet;
   the method comprising the step:
      delivering a supporting fluid via the manipulation inlet into the microchannel at or proximate to the compartment closest to the opening in the direction of flow at determined time points to dispense the compartment of sample fluid closest to the opening in the direction of flow through the opening.
74. The method of the preceding aspect, including delivering the fluid through the manipulation inlet at a manipulation volumetric flow rate Qₘₐₙᵢₚ,
   wherein Qₒᵤₜ=Qᵢₙ+Qₘₐₙᵢₚ−Q_{deposit}.
75. The method according to the preceding aspect, wherein the supporting fluid is immiscible with the sample fluid, preferably wherein the supporting fluid is the carrier fluid.
76. The method according to one of aspects 67-75, wherein the deposition device comprises a sensor unit, the method including: analyzing objects embedded in corresponding sample fluid compartments in the carrier fluid in the microchannel of the deposition device, and preferably creating corresponding sensor data.
77. The method according to the previous aspect, including using the sensor data of the deposition device to exclude sample fluid compartments from deposition, preferably by selectively inhibiting deposition of the respective compartments from the microchannel of the deposition device.
78. A method for takeup and deposition of objects, comprising the method according to any one of aspects 50 to 66 for takeup of target objects and the method according to any one of aspects 67 to 77 for depositing the target objects.
79. The method of the previous aspect, comprising:
   creating sensor data from a first or second sensor unit during the takeup of target objects;
   optionally storing the sensor data;
   using the sensor data for selective deposition of the uptaken objects.
80. The system or method according to any one of the preceding aspects, wherein the carrier fluid is a carrier liquid.
81. The system or method according to any one of the preceding aspects, wherein the sample fluid is a sample liquid.
82. The system or method according to any one of the preceding aspects, wherein the support fluid is a support liquid.
83. The system or method according to any one of the preceding aspects, wherein the microscopic objects have a size of less than 1000 μm, preferably less than 500 μm, more preferably less than 100 μm, more preferably less than 50 μm.
84. The system or method according to any one of the preceding aspects, wherein the microchannel has a cross section of at least 3000000 μm², at least 785000 μm², or at least 30000 μm².
85. The system or method according to any one of the preceding aspects, wherein the microchannel has a cross section of less than 3000000 μm², less than 785000 μm², or less than 30000 μm².
86. The system or method according to any one of the preceding aspects, wherein the microchannel has a radius of at least 1000 μm, at least 500 μm, or at least 100 μm, wherein for non-circular cross section shapes, the radii refer to the largest, the smallest or the mean radii.
87. The system or method according to any one of the preceding aspects, wherein the microchannel has a radius of less than 1000 μm, less than 500 μm, or less than 100 μm, wherein for non-circular cross section shapes, the radii refer to the largest, the smallest or the mean radii.
88. The system or method according to any one of the preceding aspects, wherein the microscopic objects are droplets, vesicles, solid particles and/or gel particles, particularly microbeads, cells, particularly animal cells or insect cells and plant cells, microorganisms, plant seeds, plant pollen, animal eggs, particularly insect eggs, spermatozoon, fungi spores, and/or embryos.
89. The system or method according to any one of the preceding aspects, wherein the opening has a size of at least 785000 μm², at least 200000 μm², or at least 8000 μm².
90. The system or method according to any one of the preceding aspects, wherein the opening has a size of less than 785000 μm², less than 200000 μm², or less than 8000 μm².
91. The system or method according to any one of the preceding aspects, wherein the opening has a radius of at least 500 μm, at least 250 μm, or at least 50 μm, wherein for non-circular cross section shapes, the radii refer to the largest, the smallest or the mean radii.
92. The system or method according to any one of the preceding aspects, wherein the opening has a radius of less than 500 μm, less than 250 μm, or less than 60 μm, wherein for non-circular cross section shapes, the radii refer to the largest, the smallest or the mean radii.
93. The system or method according to any one of the preceding aspects, wherein the microscopic objects sediment in the sample fluid, preferably under gravity of planet Earth.
94. The system or method according to any one of the preceding aspects, wherein the carrier liquid is immiscible with the sample fluid.
95. The system or method according to any one of the preceding aspects, wherein the sample liquid is as a hydrophilic liquid, preferable one or a combination of the following liquids: water, aqueous solution, pH-buffers, cell culture media and/or mixtures of water with polar organic solvents.
96. The system or method according to any one of the preceding aspects, wherein the carrier liquid is hydrophobic, and preferably one or a combination of the following liquids: alkanes, mineral oil, organic solvents, aromatic solvents, such as toluene, oligo- or polysiloxanes, fluorinated hydrocarbons, perfluorinated hydrocarbons such as perfluoromethyldecalin, aprotic organic solvents, non-water miscible liquids containing additives such as surfactants or modifiers adjusting the viscosity or surface tension, solutions of polymers or other substances.
97. The system or method according to any one of the preceding aspects, wherein the input volumetric flow of carrier fluid, the output volumetric flow of fluid and/or the takeup volumetric flow is/are continuous.
98. The system or method according to any one of the preceding aspects, wherein the settings and system properties are chosen such that the system may be stable, preferably wherein the geometries of the involved devices are chosen such that a perimeter of a sample fluid compartment at least fills the cross section of the microchannel.
99. The system or method according to any one of the preceding aspects, wherein the system properties and settings are chosen such that the one or more target objects are trapped in the respective sample fluid.
100. The system or method according to any one of the preceding aspects, wherein:
   The sample fluid wets the channel material less than the carrier fluid; and/or
   The sample fluid is an aqueous polar liquid, hydrophilic, water and/or an aqueous solution, the carrier fluid is a hydrophobic liquid, and the channel material is hydrophobic; and/or
   The carrier fluid is an aqueous polar liquid, hydrophilic, water and/or an aqueous solution, the sample fluid is an organic polar liquid, and the channel material is hydrophilic;
   The carrier fluid is a fluorinated hydrocarbon and the channel material is fluorophilic.
101. The system or method according to any one of the preceding aspects, wherein the manipulation fluid is miscible with the sample fluid.
102. The system or method according to any one of the preceding aspects, wherein the manipulation fluid is a hydrophilic liquid, preferable one or a combination of the following fluids: water, aqueous solution, cell culture media or aqueous solvent mixture.
103. The system or method according to any one of the preceding aspects, wherein the size of the opening is adapted to the cross section of the microchannel, preferably wherein the size of the opening may be smaller or equal to the cross section of the microchannel in the region of the opening.
104. The system or method according to any one of the preceding aspects, wherein the opening is a nozzle opening.
105. The system or method according to any one of the preceding aspects, wherein the settings and system properties are chosen such that the system is stable and/or the compartments are created at a frequency which remains constant over at least 10 seconds, at least 60 seconds, at least 10 minutes, or even longer.
106. The system or method according to any one of aspects 5-105, wherein the second sensor unit is configured to determine color, optical density, spectral absorption, transparency, fluorescence, phosphorescence, Raman resonances.
107. The system or method according to the previous aspect, wherein the second sensor unit is configured as a spectral analyzing unit (i.e. spectroscopic unit) generating spectroscopic data. Spectroscopy may comprise UV-Vis spectroscopy, fluorescence spectroscopy, phosphorescence spectroscopy, Infrared and/or Raman-Spectroscopy, and respective spectroscopic data may be created, stored and/or further processed.

### LIST OF REFERENCE SIGNS

2 system for handling microscopic objects
4 first microfluidic device
6 microchannel
8 inlet of microchannel
10 outlet of microchannel
12 opening
13 carrier fluid
14 (sample) container
16 (microscopic) object(s)
16a target object(s)
18 target objects 16a-1 to 16a-4
16b object cluster
18 sample fluid
18a compartment(s) of sample fluid
18a-1 to 18a-4 compartments of sample fluid
20 first sensor/imaging unit
22 surface of container 14
24 light source
25 light collecting means/lens/lens system/objective
26 optical element(s)
28 positioning unit
30 second sensor/imaging unit
32 light source
34 light collecting means
36 optical element
38 manipulation inlet
40 manipulation fluid
50 deposition device
52 target surface
52a target site
54 drop
56 neck
58 area(s) that is/are wettable by the sample fluid and non-wettable by the carrier fluid
60 area(s) that is/are non-wettable by the sample fluid and the carrier fluid
62 tip/protrusion
64 additional inlet
66 additional fluid

1. A system for handling dispersed microscopic objects contained in a sample fluid, comprising:
a first microfluidic device comprising a microchannel with an inlet, an outlet and an opening, the opening being located between the inlet and the outlet;
a conveying device configured to pump a carrier fluid via the inlet into the microchannel with an input volumetric flow rate (Qᵢₙ) and to remove fluid from the microchannel via the outlet with an output volumetric flow rate (Qₒᵤₜ);
wherein the cross section of the opening is configured such that, if the opening is in the sample fluid, the following conditions are fulfilled:
a) if the input volumetric flow rate (Qᵢₙ) is equal to the output volumetric flow rate (Qₒᵤₜ), i.e. Qᵢₙ=Qₒᵤₜ, no carrier fluid emerges from the opening into the sample fluid and no sample fluid enters the microchannel,
b) if the output volumetric flow rate (Qₒᵤₜ) is greater than the input volumetric flow rate (Qᵢₙ), i.e. Qₒᵤₜ>Qᵢₙ, sample fluid enters the microchannel via the opening so that it is embedded as one or more compartments of sample fluid in the flow of the carrier fluid;
wherein the system is configured for a takeup mode in which, at least intermittently, the output volumetric flow rate (Qₒᵤₜ) is greater than the input volumetric flow rate (Qᵢₙ), i.e. Qₒᵤₜ>Qᵢₙ, so that the system provides a flow of carrier fluid from the inlet through the microchannel and past the opening to the outlet and sequentially takes up one or more compartments of sample fluid into the flow of the carrier fluid with a takeup volumetric flow rate (Qₜₐₖₑᵤₚ);
wherein the system further comprises:
a first sensor unit for identifying positions of the dispersed objects in the sample fluid, including a position of a target object selected from the dispersed objects; and
a positioning unit configured to position the opening at a target position proximate the position of the target object such that the target object is drawn into the microchannel together with a certain volume of sample fluid and thereby embedded with the sample fluid in the carrier fluid as a compartment.

2. The system according to claim 1, wherein the system is configured to take up microscopic objects dispersed across a support surface, wherein the support surface is the bottom surface of a container in which the sample fluid is contained.

3. The system according to claim 1, wherein the system comprises a control unit.

4. The system according to the claim 3, wherein the control unit is configured to identify one or more target objects among the dispersed objects from first sensor data created by the first sensor unit by an object recognition algorithm.

5. The system according to claim 3, the control unit being configured to move the opening at a velocity (v_{opening}) towards the target object, wherein the velocity (v_{opening}) is selected such that the takeup volumetric flow rate (Qₜₐₖₑᵤₚ) compensates or substantially compensates for a displacement of sample fluid towards the target object caused by moving the first microfluidic device through the sample fluid.

6. The system according to claim 3, wherein the control unit is configured to, at least intermittently, change a velocity at which the opening is moved (v_{opening}) between a plurality of consecutive target objects during the takeup mode, wherein the velocity is adjusted according to a distance between each pair of consecutive target objects.

7. The system according to claim 3, wherein the control unit is configured to, at least intermittently, change a travel path of the opening between a pair of consecutive target objects during the takeup mode, wherein the travel path is adjusted according to a distance between the pair of target objects.

8. The system according to claim 3,
wherein the microchannel comprises one or more additional inlets for additionally providing a fluid via the one or more additional inlets to the microchannel at an additional volumetric flow rate (Q_{add}); and
wherein the control unit is configured, during takeup mode, to intermittently change the additional volumetric flow rate (Q_{add}) to be essentially equal to the absolute value of the difference (|Qᵢₙ−Qₒᵤₜ|) of the input volumetric flow rate (Qᵢₙ) and the output volumetric flow rate (Qₒᵤₜ) to prevent takeup of sample fluid through the opening.

9. The system according to claim 3, wherein the control unit is configured to intermittently vary the input volumetric flow rate (Qᵢₙ) during takeup mode in order to prevent the formation of one or more empty compartments of sample fluid when the opening is not proximate to a particle or one or more particles proximate to the opening are not to be drawn into the microchannel.

10. The system according to claim 1, wherein the microchannel comprises a manipulation inlet arranged at the opening or between the opening and the outlet, and wherein the system is configured to deliver a manipulation fluid via the manipulation inlet into a respective compartment in the microchannel at a manipulation volumetric flow rate (Qₘₐₙᵢₚ) wherein the fluid delivered via the manipulation inlet is selected from one or a combination of: a gas to be metabolized by the objects, a liquid containing a growth stimulator for the objects, a liquid containing one or more type of bacteria, a liquid containing culture media composed of nutrition and pH-Buffer components, a liquid containing an enzyme, a liquid containing ingredients for digestion of the object, parts of the object or liberation of surface bound molecules, a liquid containing compounds for analytical assay purposes, a liquid containing a PCR mix, a liquid containing antibodies, a liquid containing nanomaterials for surface-enhanced Raman spectroscopy, a liquid containing particles, and a liquid containing marker substances.

11. The system according to claim 1, wherein the system comprises a container forming a support surface, wherein the system is configured to take up the microscopic objects when dispersed across the support surface, wherein the support surface comprises a structure for prearranging the microscopic objects, the structure comprising a plurality of wells.

12. The system according to claim 1, the system comprising a storage configured to accommodate the target object(s) while embedded with the sample fluid in the carrier fluid for one or more of storage, cultivation and further treatment of the target object(s), wherein the one or more of storage, cultivation and further treatment of the target object(s) occurs within a microchannel.

13. The system according to claim 1, wherein the system comprises a microfluidic deposition device for depositing the one or more target objects at target sites, wherein the deposition device comprises a microchannel with an opening and at least one inlet, wherein a plurality of compartments of sample fluid embedded in a carrier fluid are flown via the inlet to the opening, wherein at least some of the compartments of sample fluid are dispensed through the opening and deposited on a target surface.

14. The system according to claim 1, wherein
the system is configured to deliver a support fluid via a manipulation inlet of the deposition device into the microchannel of the deposition device to squeeze a compartment of sample fluid located proximate to the opening of the deposition device out of the microchannel of the deposition device through the opening.

15. A method for picking microscopic objects by suction, comprising the steps:
a) providing a sample of dispersed microscopic objects contained in a sample fluid;
b) providing a first microfluidic device comprising a microchannel with an inlet, an outlet and an opening located between the inlet and the outlet;
c) providing a carrier fluid that is immiscible with the sample fluid via the inlet to the microchannel at an input volumetric flow rate (Qᵢₙ) and removing fluid from the microchannel via the outlet at an output volumetric flow rate (Qₒᵤₜ);
d) positioning the opening in the sample fluid;
e) setting the input volumetric flow rate (Qᵢₙ) to be smaller than the output volumetric flow rate (Qₒᵤₜ), the difference resulting in a takeup volumetric flow rate (Qₜₐₖₑᵤₚ) of sample fluid through the opening that results in compartments of sample fluid embedded in the carrier fluid;
f) changing a position of the opening relative to a target object from a starting relative position to a target position to bring the opening proximate to the target object;
g) drawing the target object into the microchannel together with a respective compartment of sample fluid when embedding the compartment of sample fluid in the carrier fluid according to step e).

16. The system according to claim 2, wherein the system comprises the container.

17. The system according to claim 1, wherein the system comprises a container forming a support surface, wherein the system is configured to take up the microscopic objects when dispersed across the support surface, wherein the support surface comprises a structure for prearranging the microscopic objects, the structure comprising one or more elongated grooves.

18. The system according to claim 1, wherein the system is configured to deliver a support fluid via a manipulation inlet of the deposition device into the microchannel of the deposition device at determined time points to squeeze a compartment of sample fluid located proximate to the opening of the deposition device out of the microchannel of the deposition device through the opening.

19. The system according to claim 1, wherein the system comprises a target surface for depositing the object(s), wherein the target surface comprises areas that are more wettable by the sample fluid than by the carrier fluid.
